(19) United States
(12) Patent Application Publication
(10) Pub. No.: US 2024/0425485 A1
(43) Pub. Date: Dec. 26, 2024

(54) PLINABULIN COMPOSITIONS

(71) Applicant: BeyondSpring Pharmaceuticals, Inc., Florham Park, NJ (US)

(72) Inventors: Lan Huang, New York, NY (US); Aniruddh Singh, New York, NY (US)

(21) Appl. No.: 18/671,711

(22) Filed: May 22, 2024

Related U.S. Application Data
(63) Continuation of application No. 17/675,453, filed on Feb. 18, 2022, now Pat. No. 12,024,501.
Application No. 17/675,453 is a continuation of application No. 16/780,780, filed on Feb. 3, 2020, now Pat. No. 11,254,657.
Application No. 16/780,780 is a continuation of application No. 16/212,386, filed on Dec. 6, 2018, now Pat. No. 10,550,104.
Application No. 16/212,386 is a continuation of application No. 15/741,635, filed on Jan. 3, 2018, now Pat. No. 10,155,748.
(60) Provisional application No. 62/191,990, filed on Jul. 13, 2015.

(51) Int. Cl. C07D 403/06; B01D 9/00

(52) U.S. Cl. CPC C07D 403/06; B01D 9/0045; B01D 9/0054; B01D 9/0063; B01D 2009/0086; C07B 2200/13

(57) ABSTRACT

Disclosed herein are plinabulin polymorphs, compositions, their use and preparation as therapeutic agents. In particular, some embodiments relate to plinabulin monohydrate in a crystalline form.

### INCORPORATION BY REFERENCE TO ANY PRIORITY APPLICATIONS

This application is a continuation of U.S. application Ser. No. 17/675,453 filed on Feb. 18, 2022, which is a continuation application of U.S. application Ser. No. 16/780,780 filed on Feb. 3, 2020, which is the continuation application of U.S. application Ser. No. 16/212,386 filed on Dec. 6, 2018, which is the continuation application of U.S. application Ser. No. 15/741,635 filed on Jan. 3, 2018, which is the U.S. National Phase of International Application No. PCT/US2016/041773 entitled PLINABULIN COMPOSITIONS, filed Jul. 11, 2016 and published on Jan. 19, 2017 as WO 2017/011399, which claims the benefit of U.S. Provisional Application No. 62/191,990, filed Jul. 13, 2015, the disclosure of which are incorporated herein by reference in their entireties.

### BACKGROUND

### Field

The present invention relates to the fields of chemistry and medicine. More particularly, the present invention relates to forms and compositions of plinabulin and their preparation.

### Description of the Related Art

Plinabulin is a synthetic analog of diketopiperazine phenylahistin (halimide) discovered from marine and terrestrial Aspergillus sp. Plinabulin is structurally different from colchicine and its combretastatin-like analogs (eg, fosbretabulin) and binds at or near the colchicine binding site on tubulin monomers. Previous studies showed that plinabulin induced vascular endothelial cell tubulin depolymerization and monolayer permeability at low concentrations compared with colchicine and that it induced apoptosis in Jurkat leukemia cells. Studies of plinabulin as a single agent in patients with advanced malignancies (lung, prostate, and colon cancers) showed a favorable pharmacokinetic, pharmacodynamics, and safety profile.

### SUMMARY OF THE INVENTION

Some embodiments relate to a plinabulin monohydrate.

Other embodiments relate to a plinabulin monohydrate in crystalline form.

Some embodiments relate to a plinabulin composition having more than about 90% by weight of plinabulin, based on the total weight of the composition.

Other embodiments relate to a plinabulin composition having more than about 99% by weight of plinabulin, based on the total weight of molecules in the composition other than water, dimethylformamide, ethanol, ethyl acetate, methanol, toluene, and acetic acid.

Some embodiments relate to a plinabulin composition, containing plinabulin and no more than about 1.9% by weight of impurities, based on the total weight of the composition other than water.

Other embodiments relate to a plinabulin composition, containing plinabulin and no more than about 1% by weight of impurities other than solvent molecules, based on the total weight of non-solvent molecules in the composition.

Some embodiments relate to a plinabulin composition having plinabulin and no more than about 1% by weight of impurities, based on a HPLC analysis.

Some embodiments relate to a process of preparing a plinabulin monohydrate or plinabulin composition.

### BRIEF DESCRIPTION OF THE DRAWINGS

FIG. 1 is an X-ray powder diffraction (XRPD) pattern of the crystalline form of plinabulin monohydrate.

FIG. 2 shows a polarized light microscopy (PLM) image of a sample containing the crystalline form of plinabulin monohydrate.

FIG. 3A shows the thermo gravimetric (TGA) of the crystalline form of plinabulin monohydrate (Form 1); FIG. 3B shows the digital scanning calorimetry (DSC) analysis results of the crystalline form of plinabulin monohydrate (Form 1); and FIG. 3C shows the Dynamic Vapor Sorption (DVS) isotherm plot.

FIG. 4 is an XRPD pattern of the crystalline form 2.

FIG. 5 shows a PLM image of a sample containing the crystalline form 2.

FIG. 6A shows the TGA analysis of the crystalline form 2 and FIG. 6B shows the DSC analysis results of the crystalline form 2.

FIG. 7 is an XRPD pattern of the crystalline form 3.

FIG. 8 shows a PLM image of a sample containing the crystalline form 3.

FIG. 9A shows the TGA analysis of the crystalline form 3; and

FIG. 9B shows the DSC analysis results of the crystalline form 3.

FIG. 10 is an XRPD pattern of the crystalline form 4.

FIG. 11 shows a PLM image of a sample containing the crystalline form 4.

FIG. 12A shows the TGA analysis of the crystalline form 4; and

FIG. 12B shows the digital scanning calorimetry (DSC) analysis results of the crystalline form 4.

FIG. 13 is an XRPD pattern of the crystalline form 5.

FIG. 14 shows the TGA analysis of the crystalline form 5

FIG. 15 shows the DSC analysis results of the crystalline form 5.

FIG. 16 is an XRPD pattern of the crystalline form 6.

FIG. 17 shows the TGA analysis of the crystalline form 6

FIG. 18 shows the DSC analysis results of the crystalline form 6.

FIG. 19 is an XRPD pattern of the crystalline form 7.

FIG. 20 shows the TGA of the crystalline form 7

FIG. 21 shows the DSC analysis results of the crystalline form 7.

FIG. 22 is an XRPD pattern of the crystalline form 8.

FIG. 23 shows the TGA of the crystalline form 8.

FIG. 24 shows the DSC analysis results of the crystalline form 8.

FIG. 25 is an XRPD pattern of the crystalline form 9.

FIG. 26 shows the TGA analysis of the crystalline form 9

FIG. 27 shows the DSC analysis results of the crystalline form 9.

FIG. 28 shows the inter-conversion of plinabulin polymorph forms.

FIG. 29 is a flow diagram of preparing the plinabulin monohydrate composition.

### DETAILED DESCRIPTION OF THE PREFERRED EMBODIMENT

Plinabulin, (3Z,6Z)-3-Benzylidene-6-{[5-(2-methyl-2-propanyl)-1H-imidazol-4-yl]methylene}-2,5-piperazinedione, is a synthetic analog of the natural compound phenylahistin. Plinabulin can be readily prepared according to methods and procedures detailed in U.S. Pat. Nos. 7,064,201 and 7,919,497, which are incorporated herein by reference in their entireties. Some embodiments relate to polymorphs and solvates (e.g., hydrates) of plinabulin and pharmaceutical compositions comprising the same. Some embodiments include methods of preparation and methods of treatment. In particular, some embodiments relate to a plinabulin monohydrate.

### Plinabulin Monohydrate

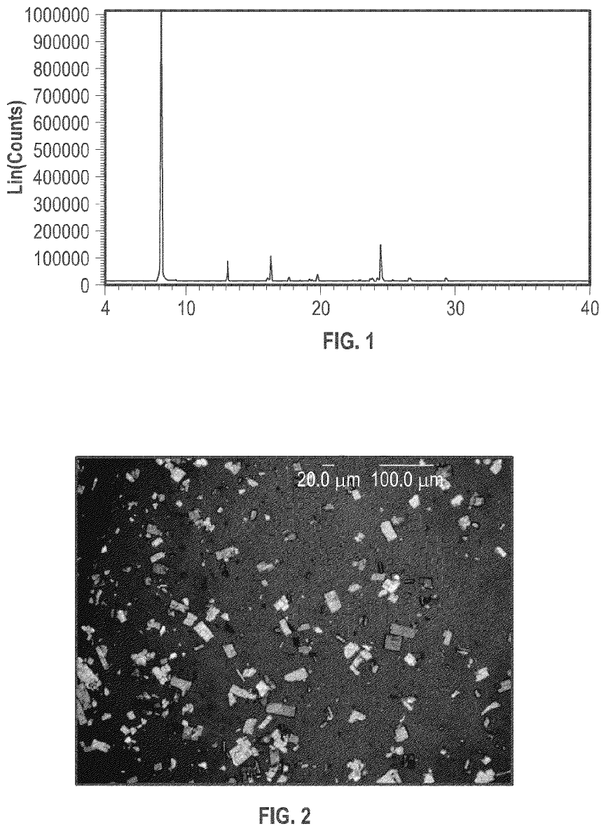

Plinabulin monohydrate (Form 1) is stable crystalline form of plinabulin. The X-ray powder diffraction (PXRD) pattern of plinabulin monohydrate (Form 1) is substantially the same as shown in FIG. 1, with corresponding tabulated peak data shown in Table 1.

TABLE 1
Peak Data of X-ray powder diffraction (PXRD) pattern
of plinabulin monohydrate (Form 1)

| Angle (2-θ) | Intensity(%) | D value (angstrom) |
|---|---|---|
| 8.14 | 100.0 | 10.854 |
| 11.16 | 6.2 | 7.923 |
| 13.08 | 19.7 | 6.764 |
| 13.91 | 4.9 | 6.363 |
| 14.13 | 5.3 | 6.263 |
| 14.83 | 6.2 | 5.969 |
| 15.50 | 5.2 | 5.714 |
| 16.06 | 8.1 | 5.515 |
| 16.29 | 13.4 | 5.437 |
| 17.64 | 7.2 | 5.023 |
| 18.47 | 4.9 | 4.799 |
| 19.17 | 6.8 | 4.627 |
| 19.35 | 6.2 | 4.583 |
| 19.79 | 8.9 | 4.482 |
| 20.88 | 5.6 | 4.251 |
| 22.42 | 6.2 | 3.963 |
| 22.87 | 8.4 | 3.886 |
| 23.87 | 13.4 | 3.726 |
| 24.23 | 14.4 | 3.670 |
| 24.53 | 17.0 | 3.626 |
| 25.38 | 8.2 | 3.506 |
| 26.59 | 10.8 | 3.350 |
| 27.19 | 4.8 | 3.277 |
| 27.44 | 5.5 | 3.248 |
| 27.95 | 4.8 | 3.190 |
| 28.90 | 5.0 | 3.087 |
| 29.34 | 9.5 | 3.041 |

In some embodiments, the plinabulin monohydrate (Form 1) described herein includes a crystalline form exhibiting an X-ray powder diffraction pattern comprising at least three characteristic peaks selected from the group consisting of peaks at approximately 8.1°, 13.1°, 16.3°, 23.9°, 24.2°, 24.5°, and 26.6° 2θ. In some embodiments, the plinabulin monohydrate (Form 1) described herein includes a crystalline form exhibiting an X-ray powder diffraction pattern comprising at least peaks at approximately 8.1°, 13.1°, 16.3°, 23.9°, 24.2°, 24.5°, and 26.6° 2θ. In some embodiments, the plinabulin monohydrate (Form 1) described herein includes a crystalline form exhibiting an X-ray powder diffraction pattern comprising at least peaks at approximately 8.1°, 13.1°, 16.1°, 16.3°, 19.8°, 22.9°, 23.9°, 24.2°, 24.5°, 26.6°, and 29.3° 2θ.

As is well understood in the art, because of the experimental variability when X-ray diffraction patterns are measured on different instruments, the peak positions are assumed to be equal if the two theta (2θ) values agree to within 0.2° (i.e., ±0.2°). For example, the United States Pharmacopeia states that if the angular setting of the 10 strongest diffraction peaks agree to within ±0.2° with that of a reference material, and the relative intensities of the peaks do not vary by more than 20%, the identity is confirmed. Accordingly, peak positions within 0.2° of the positions recited herein are assumed to be identical. Unless otherwise indicated, all X-ray diffraction angles recited herein are based on a copper K-alpha source.

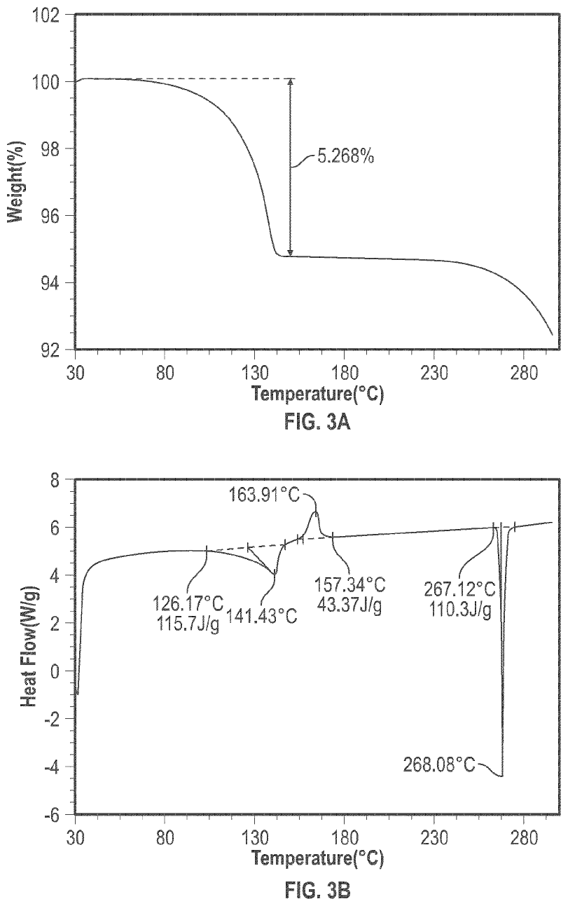

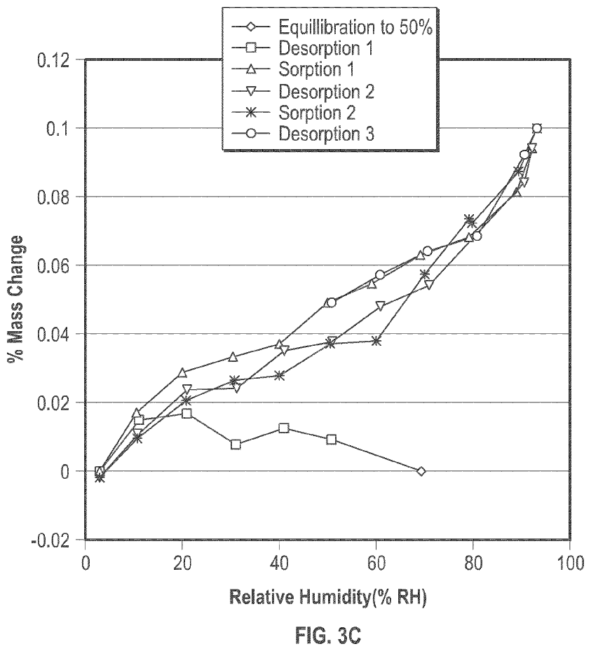

FIG. 3B shows digital scanning calorimetry (DSC) analysis results of the crystalline form of the plinabulin monohydrate (Form 1). As shown in FIG. 3B, the crystalline form of the plinabulin monohydrate (Form 1) has a melting point of about 267° C.; the crystalline form of the plinabulin monohydrate (Form 1) has a differential scanning calorimetry thermogram with endothermic peaks at about 141° C. and about 267° C.

The crystalline form of the plinabulin monohydrate (Form 1) is more stable than the other polymorph forms. The plinabulin monohydrate (Form 1) can remain stable during the DVS and drying tests as compared to other polymorph forms, which may show weight change and degradation during the tests.

### Plinabulin Composition

Some embodiments relate to a plinabulin composition that includes more than about 50% by weight of the plinabulin monohydrate (Form 1) described herein, based on the total weight of the composition. In some embodiments, the plinabulin composition includes more than about 75% of the plinabulin monohydrate (Form 1) described herein. In some embodiments, the plinabulin composition includes more than about 90% of the plinabulin monohydrate described herein. In some embodiments, the plinabulin composition includes more than about 95% of the plinabulin monohydrate described herein. In some embodiments, the plinabulin composition includes more than about 98% of the plinabulin monohydrate described herein. In some embodiments, the plinabulin composition includes more than about 99% of the plinabulin monohydrate described herein. In some embodiments, the plinabulin composition includes the plinabulin monohydrate described herein in the range of about 50% to about 99%, about 60% to about 99%, about 70% to about 99%, about 80% to about 99%, about 90% to about 99%, about 95% to about 99%, or about 97.5% to about 99%, based on the total weight of the composition. The remaining portion of the plinabulin composition may be other forms of plinabulin and/or other chemical entities.

Some embodiments relate to a plinabulin composition with a high purity. In particularly, some embodiments relate to a plinabulin composition having more than about 90% by weight of plinabulin, based on the total weight of the composition. In some embodiments, the plinabulin composition includes more than about 92% of the plinabulin compound. In some embodiments, the plinabulin composition includes more than 80%, 85%, 86%, 87%, 88%, 89%, 90%, 91,%, 92%, 93%, 94%, 95%, 96%, 96.5%, 96%, 98%, 99%, or 99.6% of the plinabulin compound. In some embodiments, the plinabulin composition includes more than about 99% by weight of plinabulin, based on the total weight of non-solvent molecules in the composition. In some embodiments, the plinabulin composition includes more than about 96%, 97%, 98%, 99%, or 99.6% by weight of plinabulin, based on the total weight of non-solvent molecules in the composition. In some embodiments, the solvent can be water, dimethylformamide, ethanol, ethyl acetate, methanol, toluene, and acetic acid. In some embodiments, the plinabulin in the high-purity composition is present, at least in part, in plinabulin monohydrate as described above.

In some embodiments, the plinabulin composition includes more than about 99% by weight of a plinabulin, based on the total weight of molecules in the composition other than water, dimethylformamide, ethanol, ethyl acetate, methanol, toluene, and acetic acid. In some embodiments, the plinabulin composition includes more than about 80%, 85%, 86%, 87%, 88%, 89%, 90%, 91,%, 92%, 93%, 94%, 95%, 96%, 96.5%, 96%, 98%, 99%, or 99.6% by weight of a plinabulin, based on the total weight of molecules in the composition other than water, dimethylformamide, ethanol, ethyl acetate, methanol, toluene, and acetic acid. In some embodiments, the plinabulin in the composition is present, at least in part, in plinabulin monohydrate as described above.

In some embodiments, the plinabulin composition includes more than about 99% by weight of a plinabulin, based on a HPLC analysis. In some embodiments, the plinabulin composition includes more than about 80%, 85%, 86%, 87%, 88%, 89%, 90%, 91,%, 92%, 93%, 94%, 95%, 96%, 96.5%, 96%, 98%, 99%, or 99.6% by weight of a plinabulin, based on a HPLC analysis. In some embodiments, the plinabulin in the composition is present, at least in part, in plinabulin monohydrate as described above.

Some embodiments relate to a plinabulin composition with low levels of impurities. The term “impurity” as used herein refers to one or more components of the composition that is different from plinabulin and water. In some embodiments, the impurity can include one or more chemical compounds introduced during the synthesis of plinabulin. In some embodiments, the impurity can include dimethylformamide, ethanol, ethyl acetate, methanol, toluene, acetic acid, and other residual solvent.

In some embodiments, the plinabulin composition includes no more than about 1% by weight of impurities, based on the total weight of the composition. In some embodiments, the plinabulin composition includes no more than about 15%, 14%, 13%, 12%, 11%, 10%, 9%, 8%, 7%, 6%, 5%, 4%, 3%, 2%, 1%, 0.97%, 0.9%, 0.8%, 0.6%, 0.4% or 0.2% by weight of impurities, based on the total weight of the composition. In some embodiments, the plinabulin composition includes no more than about 1% by weight of impurities, based on the total weight of non-solvent molecules in the composition. In some embodiments, the plinabulin composition includes no more than about 15%, 14%, 13%, 12%, 11%, 10%, 9%, 8%, 7%, 6%, 5%, 4%, 3%, 2%, 1%, 0.97%, 0.9%, 0.8%, 0.6%, 0.4% or 0.2% by weight of impurities, based on the total weight of non-solvent molecules in the composition.

In some embodiments, the plinabulin composition includes no more than about 1.9% by weight of impurities, based on the total weight of the composition other than water. In some embodiments, the plinabulin composition includes no more than about 15%, 14%, 13%, 12%, 11%, 10%, 9%, 8%, 7%, 6%, 5%, 4%, 3%, 2%, 1%, 0.97%, 0.9%, 0.8%, 0.6%, 0.4% or 0.2% by weight of impurities, based on the total weight of the composition other than water. In some embodiments, the plinabulin composition includes no more than about 1% by weight of impurities, based on the total weight of non-solvent molecules in the composition other than water. In some embodiments, the plinabulin composition includes no more than about 15%, 14%, 13%, 12%, 11%, 10%, 9%, 8%, 7%, 6%, 5%, 4%, 3%, 2%, 1%, 0.97%, 0.9%, 0.8%, 0.6%, 0.4% or 0.2% by weight of impurities, based on the total weight of non-solvent molecules in the composition other than water.

In some embodiments, the plinabulin composition includes no more than about more than about 0.9% by weight of impurities other than dimethylformamide, ethanol, ethyl acetate, methanol, toluene, and acetic acid, based on the total weight of molecules in the composition other than water, dimethylformamide, ethanol, ethyl acetate, methanol, toluene, and acetic acid. In some embodiments, the plinabulin composition includes no more than about more than about 5%, 4%, 3%, 2%, 1%, 0.97%, 0.9%, 0.8%, 0.6%, 0.4% or 0.2% by weight of impurities other than dimethylformamide, ethanol, ethyl acetate, methanol, toluene, and acetic acid, based on the total weight of molecules in the composition other than water, dimethylformamide, ethanol, ethyl acetate, methanol, toluene, and acetic acid.

In some embodiments, the plinabulin composition includes no more than about more than about 1% by weight of impurities, based on a HPLC analysis. In some embodiments, the plinabulin composition includes no more than about more than about 5%, 4%, 3%, 2%, 1%, 0.97%, 0.9%, 0.8%, 0.6%, 0.4% or 0.2% by weight of impurities, based on a HPLC analysis.

### Method of Preparation

Some embodiments relate to a process of preparing the plinabulin monohydrate or the plinabulin composition described herein, the method including: combining plinabulin and a first solvent system to form a first mixture, heating the first mixture to a temperature in the range of about 50° C. to 90° C., and cooling the first mixture to form a first precipitate.

In some embodiments, the process further includes filtering prior to cooling the first mixture. In some embodiments, the process further includes adding water to the first mixture prior to heating.

In some embodiments, the process described herein further includes filtering the first precipitate. In some embodiments, the process described herein further includes washing the first precipitate.

In some embodiments, the first solvent system can be water, alcohol, or a mixture of water and alcohol.

In some embodiments, the alcohol is selected from methanol, ethanol, isopropyl alcohol, tert-butyl alcohol and n-butyl alcohol; or mixture thereof.

In some embodiments, the alcohol is ethanol.

In some embodiments, heating the first mixture comprises refluxing the first mixture.

In some embodiments, the first mixture is heated to about 70° C. to 78° C. In some embodiments, the first mixture is heated to about 60° C. to 90° C. In some embodiments, the first mixture is heated to about 60° C. to 80° C. In some embodiments, the first mixture is heated to the boiling point of ethanol.

In some embodiments, the process described herein further includes maintaining the first mixture at a refluxing temperature for about 1 hour prior to cooling the first mixture.

In some embodiments, heating the first mixture includes heating the first mixture to at least 65° C., and wherein cooling the first mixture includes cooling the first mixture to about 50° C. to 60° C.

In some embodiments, the cooling of the first mixture includes adding water to the first mixture to produce the first precipitate.

In some embodiments, the cooling of the first mixture includes stirring the first mixture for at least 4 hours.

In some embodiments, the process described herein further includes analyzing the first precipitate to determine the plinabulin composition in the first precipitate.

In some embodiments, the process described herein further includes combining the first precipitate and a second solvent to form a second mixture and heating the second mixture to a temperature in the range of about 50° C. to 90° C.; cooling the second mixture to form a second precipitate; and filtering the second precipitate and washing the second precipitate.

In some embodiments, the second solvent is water, alcohol, or a mixture of water and alcohol.

In some embodiments, the alcohol is selected from methanol, ethanol, isopropyl alcohol, tert-butyl alcohol and n-butyl alcohol; or mixture thereof. In some embodiments, the alcohol is ethanol.

In some embodiments, heating the second mixture comprises refluxing the second mixture.

In some embodiments, the second mixture is heated to about 70° C. to 78° C.

In some embodiments, the process described herein includes maintaining the second mixture at a refluxing temperature for about 1 hour prior to cooling the second mixture.

In some embodiments, cooling the second mixture comprises cooling the first mixture to about 15° C. to 30° C.

In some embodiments, the cooling of the second mixture comprises adding water to the second mixture to produce the second precipitate.

In some embodiments, the cooling of the second mixture comprises stirring the second mixture for at least 4 hours.

In some embodiments, the first precipitate is washed with an alcohol and the washed alcohol is collected and added to the second mixture prior to the heating.

In some embodiments, the process described herein includes drying the second precipitate

In some embodiments, the process described herein includes analyzing the second precipitate to determine the plinabulin composition in the second precipitate.

In some embodiments, the combining, cooling, and filtering steps are repeated one or more times based on the plinabulin composition in the second precipitate.

Some embodiments relate to a process of preparing the plinabulin monohydrate or the plinabulin composition, wherein the process includes mixing plinabulin, ethanol, and water to form a mixture. In some embodiments, the process includes the mixture.

In some embodiments, the volume ratio of the ethanol to water is about 95:5. In some embodiments, the volume ratio of the ethanol to water is about 85:15, 90:10, 95:5, 97.5:2.5, or 99:1. In some embodiments, the volume ratio of the ethanol to water is about 15, 16, 17, 18, 19, 20, 21, 22, 23, 24, or 25.

In some embodiments, the mixture of plinabulin and the solvent system is stirred for at least about 1, 2, 3, 4, 5, 6, 7, 8, 9, 10, 15, or 20 hours. In some embodiments, the mixture is stirred for at least 2 hours.

In some embodiments, the mixing or stirring is performed at a temperature in the range of about 10° C. to about 50° C.; about 20° C. to about 40° C.; about 25° C. to about 35° C., or about 28° C. to about 32° C. In some embodiments, the mixing of plinabulin, ethanol and water to form the mixture or stirring of the mixture is performed at about 20° C., 25° C., 30° C., 35° C., or 40° C.

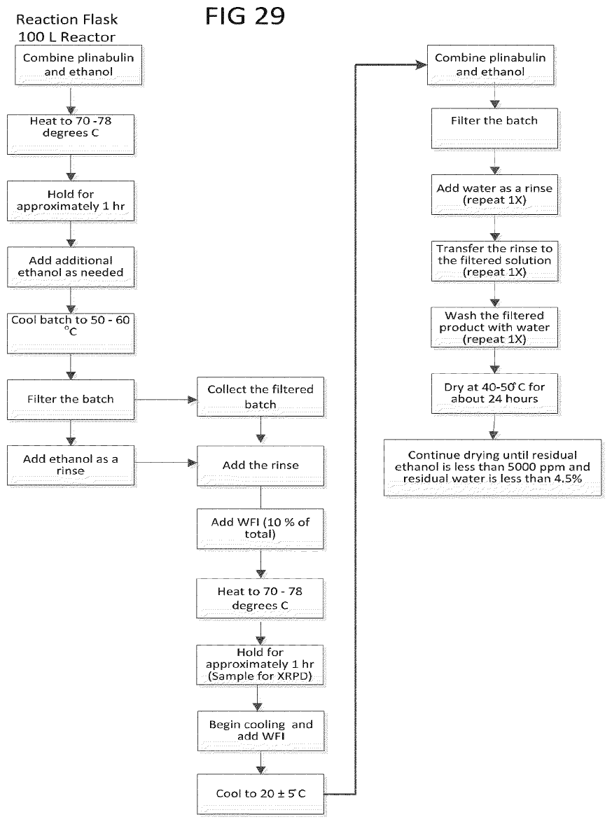

FIG. 29 shows a block diagram of one method for producing the plinabulin monohydrate form composition. As shown in FIG. 29, the plinabulin compound and ethanol are added to a reaction flask, and the mixture is then heated to 70-78° C. and mixed at this temperature for about 1 hour. Additional ethanol can be added if needed. The flask is then cooled to about 50-60° C., the mixture is filtered, and ethanol is used as a rinse solvent. The filtered solution and the rinse solution are combined and water is added to about 10% of the combined solution. The solution is then heated to 70-78° C. and mixed at this temperature for about 1 hour. A sample is taken for XRPD analysis, and then the solution is cooled to 20±5° C. and added with water. The batch is filtered and water is used as a rinse. The filtered product is washed with water and then transferred to the drying trays to dry at 40-50° C. for about 24 hours or longer until it reaches the required ethanol and water content levels.

### Plinabulin Crystalline Form 2

Some embodiments relate to a crystalline Form 2 of plinabulin and its process of preparation. While not being bound by any particular theory, it is believed that Form 2 is a plinabulin isopropyl alcohol (IPA)) solvate.

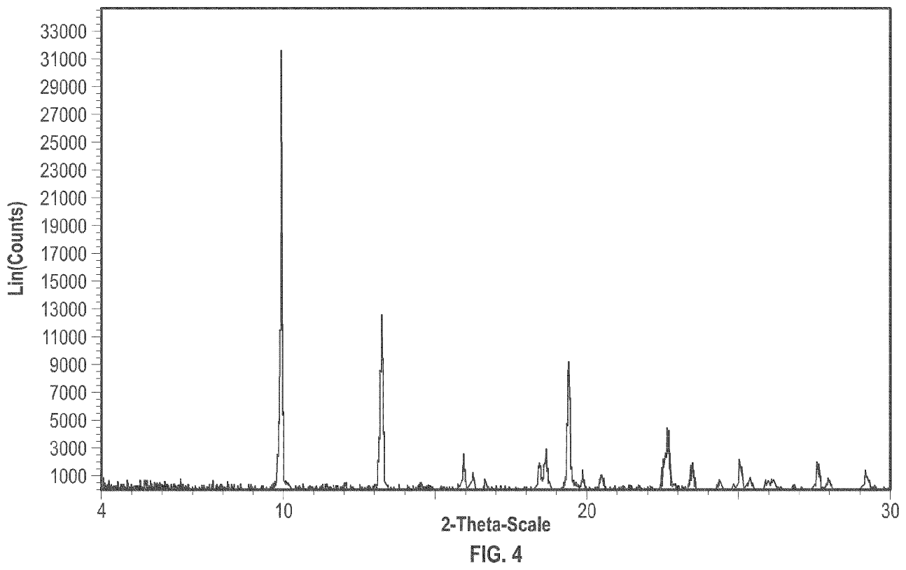

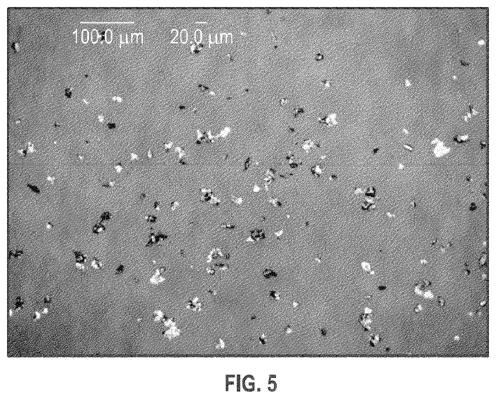

In some embodiments, crystalline Form 2 of plinabulin has substantially the same XRPD pattern as depicted in FIG. 4, with corresponding tabulated peak data shown in Table 2.

TABLE 2
Peak Data of PXRD pattern of crystalline Form 2

| Angle, 2θ | d spacing | Intensity, % |
|---|---|---|
| 9.93 | 8.90 | 100 |
| 13.25 | 6.68 | 39.6 |
| 15.96 | 5.55 | 7.9 |
| 16.28 | 5.44 | 3.7 |
| 16.69 | 5.31 | 2.2 |
| 18.47 | 4.80 | 5.5 |
| 18.68 | 4.75 | 9 |
| 19.45 | 4.56 | 28.9 |
| 19.90 | 4.46 | 4.2 |
| 20.53 | 4.32 | 3.1 |
| 22.71 | 3.91 | 14 |
| 23.51 | 3.78 | 6 |
| 24.44 | 3.64 | 2 |
| 25.12 | 3.54 | 6.7 |
| 25.45 | 3.50 | 2.6 |
| 26.06 | 3.42 | 1.4 |
| 26.19 | 3.40 | 1.9 |
| 27.69 | 3.22 | 5.9 |
| 28.04 | 3.18 | 2.3 |
| 29.27 | 3.05 | 4 |

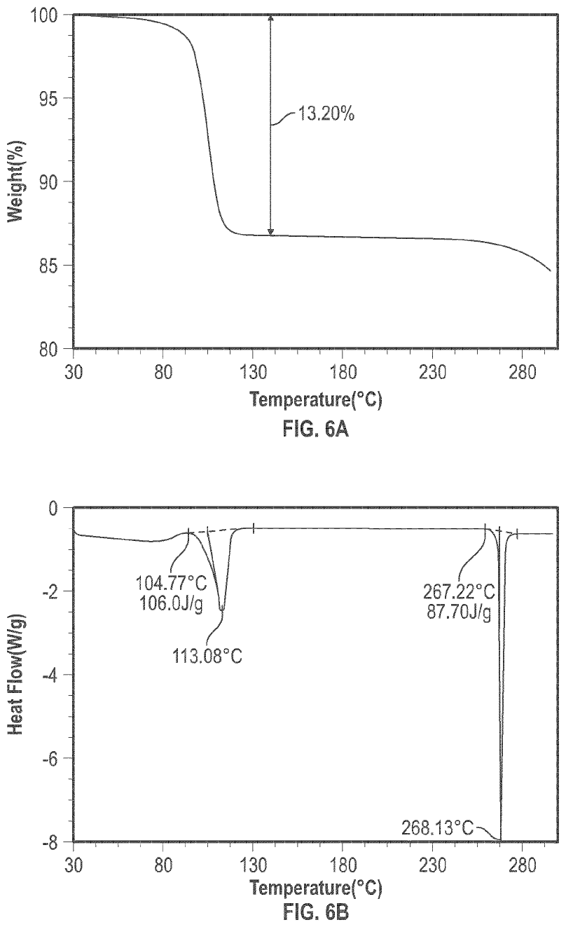

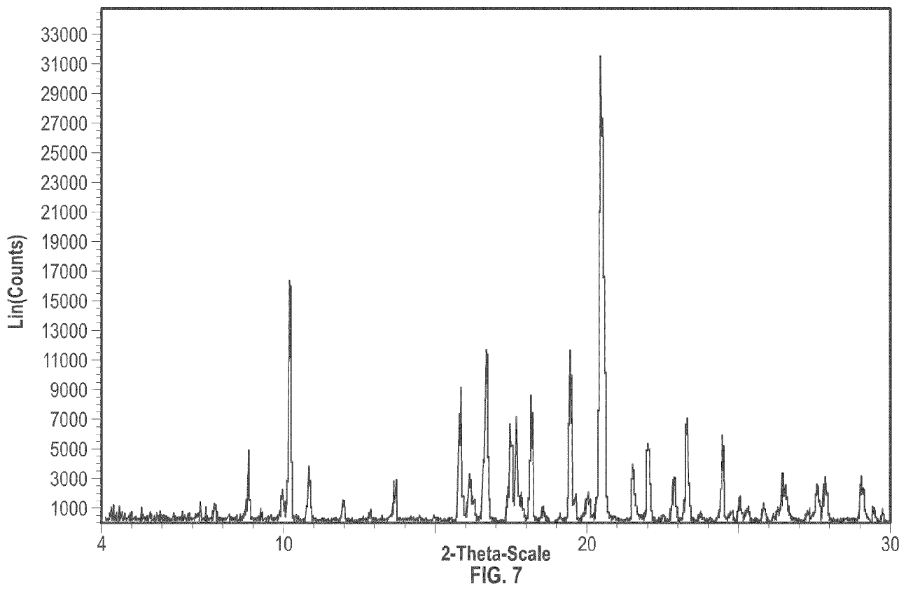

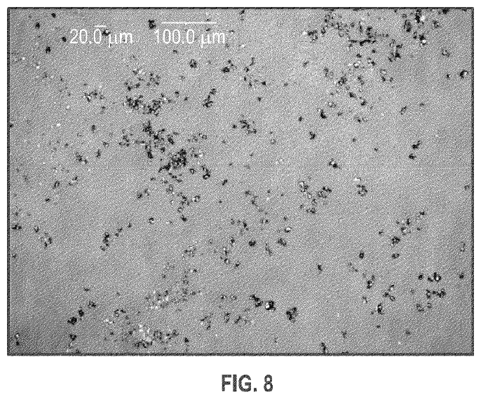

The crystalline Form 2 may further be characterized by a DSC thermogram substantially the same as that depicted in FIG. 6B. As shown in FIG. 6B, the crystalline Form 2 has a melting point of about 267° C.; the crystalline form of plinabulin Form 2 has a differential scanning calorimetry thermogram with endothermic peaks at about 113° C. and about 267° C.

### Plinabulin Crystalline Form 3

Some embodiments relate to a crystalline Form 3 of plinabulin and its process of preparation. While not being bound by any particular theory, it is believed that Form 3 is an anhydrous form of plinabulin.

In some embodiments, crystalline Form 3 of plinabulin has substantially the same XRPD pattern as depicted in FIG. 7, with corresponding tabulated peak data shown in Table 3.

TABLE 3
Peak Data of PXRD pattern of crystalline Form 3

| Angle, 2θ | d spacing | Intensity, % |
|---|---|---|
| 7.75 | 11.40 | 4.3 |
| 8.84 | 9.99 | 17.8 |
| 9.97 | 8.86 | 7.9 |
| 10.21 | 8.66 | 59.2 |
| 10.86 | 8.14 | 13.9 |
| 11.99 | 7.38 | 5.2 |
| 12.87 | 6.87 | 3.2 |
| 13.69 | 6.46 | 10.3 |
| 15.87 | 5.58 | 32.7 |
| 16.15 | 5.48 | 11.7 |
| 16.71 | 5.30 | 41.9 |
| 17.54 | 5.05 | 23.9 |
| 17.73 | 5.00 | 25.8 |
| 17.86 | 4.96 | 8 |
| 18.19 | 4.87 | 31.4 |
| 18.59 | 4.77 | 3.9 |
| 19.15 | 4.63 | 2.3 |
| 19.50 | 4.55 | 42.4 |
| 20.06 | 4.42 | 7.5 |
| 20.52 | 4.32 | 100 |
| 21.58 | 4.11 | 14.2 |
| 22.08 | 4.02 | 19.4 |
| 22.92 | 3.88 | 10.9 |
| 23.35 | 3.81 | 25.3 |
| 24.54 | 3.63 | 20.3 |
| 24.80 | 3.59 | 2.8 |
| 25.10 | 3.55 | 6.2 |
| 25.34 | 3.51 | 3.8 |
| 25.89 | 3.44 | 4.5 |
| 26.53 | 3.36 | 10.2 |
| 27.35 | 3.26 | 2.7 |
| 27.65 | 3.22 | 9.4 |
| 27.93 | 3.19 | 11.2 |
| 29.13 | 3.06 | 11.3 |
| 29.54 | 3.02 | 3.1 |
| 29.81 | 29.81 | 29.81 |

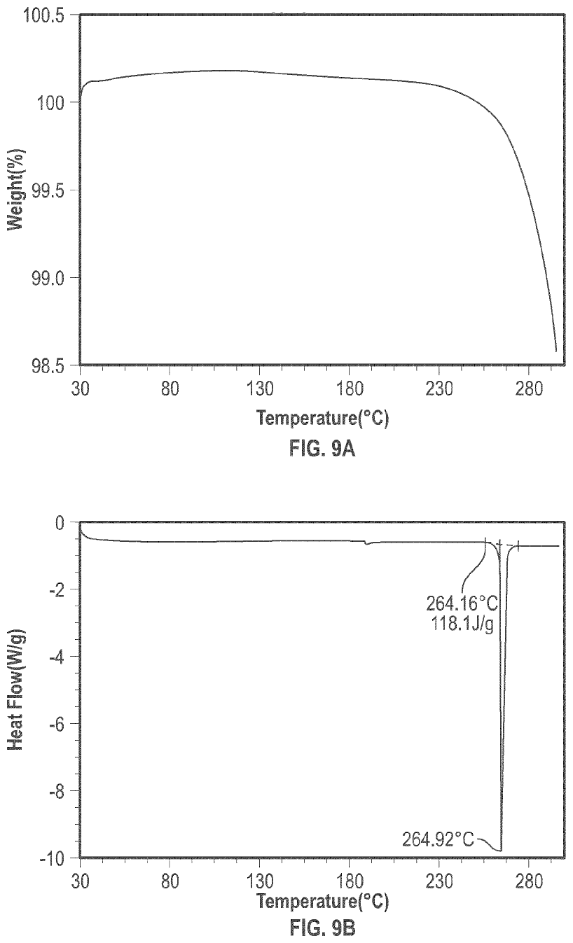

The crystalline Form 3 may further be characterized by a DSC thermogram substantially the same as that depicted in FIG. 9B. As shown in FIG. 9B, the crystalline Form 3 has a melting point of about 264° C.; the crystalline Form 3 has a differential scanning calorimetry thermogram with endothermic peak at about 264° C.

### Plinabulin Crystalline Form 4

Some embodiments relate to a crystalline Form 4 of plinabulin and its process of preparation. While not being bound by any particular theory, it is believed that Form 4 is a plinabulin methanol solvate.

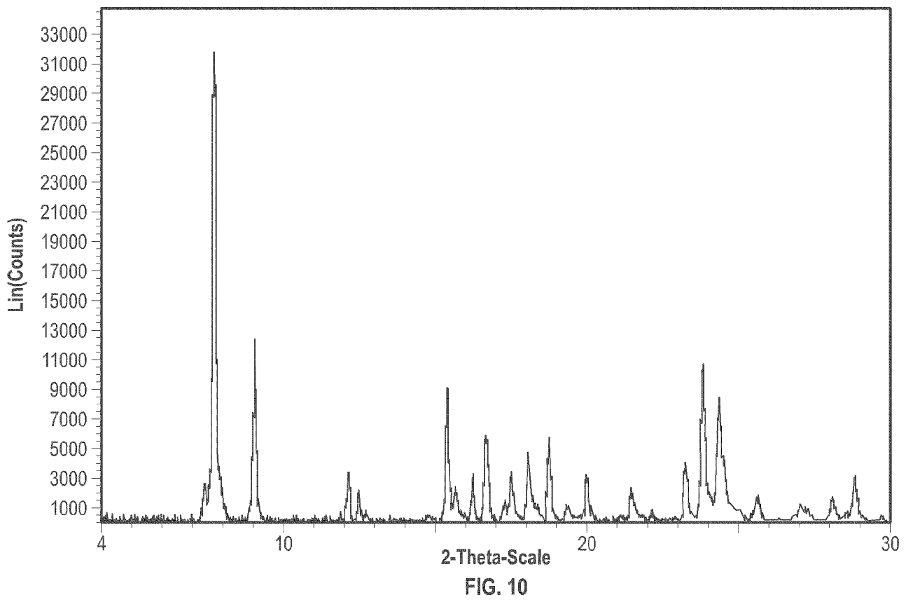

In some embodiments, crystalline Form 4 of plinabulin has substantially the same XRPD pattern as depicted in FIG. 10, with corresponding tabulated peak data shown in Table 4.

TABLE 4
Peak Data of PXRD pattern of crystalline Form 4

| Angle, 2θ | d spacing | Intensity, % |
|---|---|---|
| 7.41 | 11.93 | 7.9 |
| 7.71 | 11.45 | 100 |
| 9.06 | 9.76 | 38.6 |
| 12.16 | 7.28 | 10.2 |
| 12.50 | 7.07 | 5.6 |
| 12.74 | 6.94 | 2.2 |
| 15.44 | 5.73 | 28.2 |
| 15.70 | 5.64 | 7.2 |
| 16.27 | 5.44 | 10.1 |
| 16.72 | 5.30 | 17.4 |
| 17.33 | 5.11 | 4.6 |
| 17.56 | 5.05 | 10.5 |
| 18.13 | 4.89 | 14.6 |
| 18.79 | 4.72 | 17.5 |
| 19.39 | 4.57 | 3.5 |
| 20.03 | 4.43 | 9.6 |
| 21.53 | 4.12 | 7 |
| 23.32 | 3.81 | 12.4 |
| 23.90 | 3.72 | 33.5 |
| 24.42 | 3.64 | 26.2 |
| 25.69 | 3.47 | 5.3 |
| 27.08 | 3.29 | 3.3 |
| 28.15 | 3.17 | 4.4 |
| 28.90 | 3.09 | 9.6 |

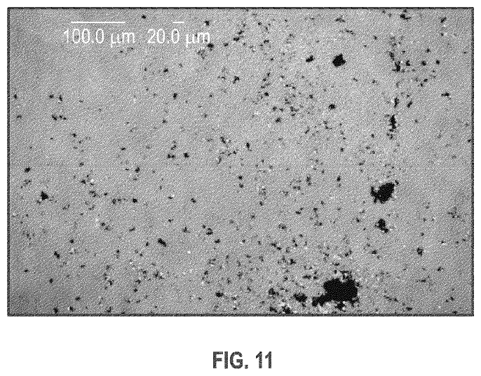

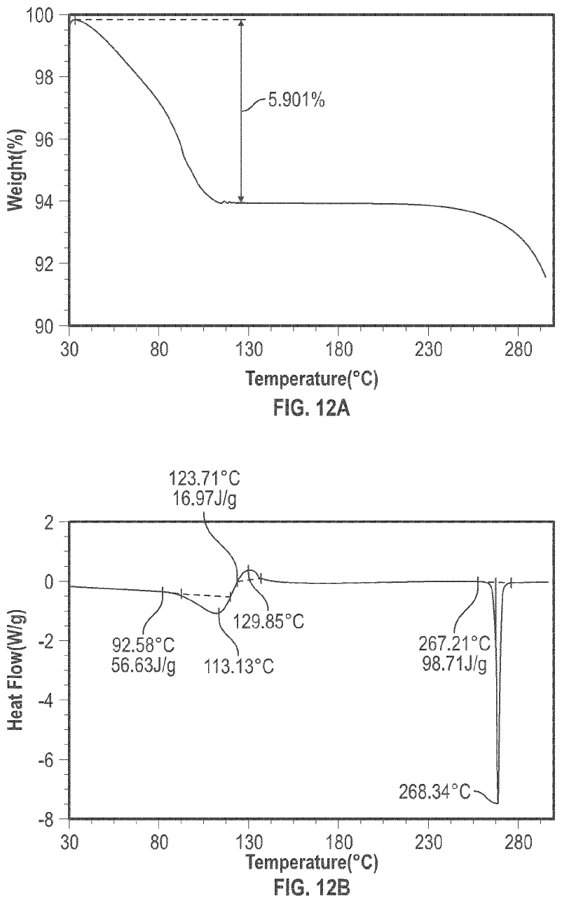

The crystalline Form 4 may further be characterized by a DSC thermogram substantially the same as that depicted in FIG. 12B. As shown in FIG. 12B, the crystalline Form 4 has a melting point of about 267° C.; the crystalline Form 4 has a differential scanning calorimetry thermogram with endothermic peaks at about 113° C. and at about 264° C.

### Plinabulin Crystalline Form 5

Some embodiments relate to a crystalline Form 5 of plinabulin and its process of preparation.

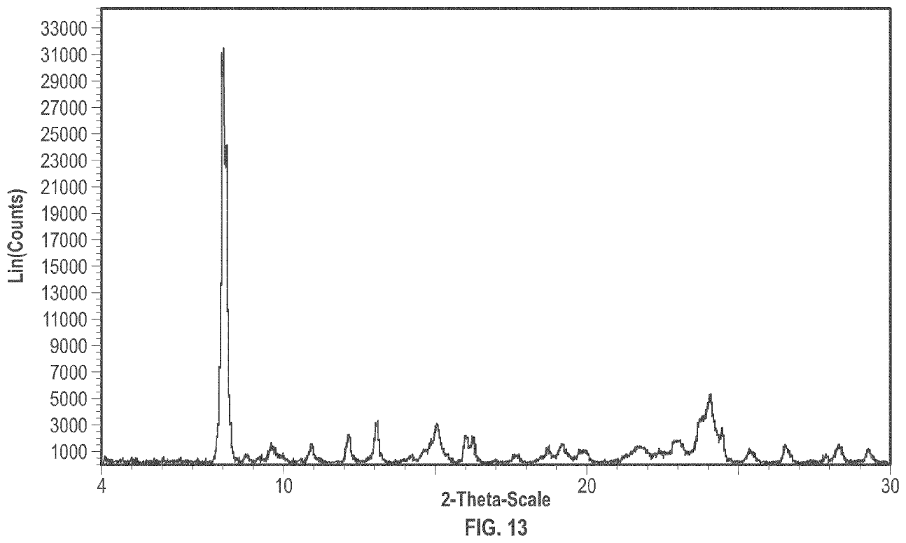

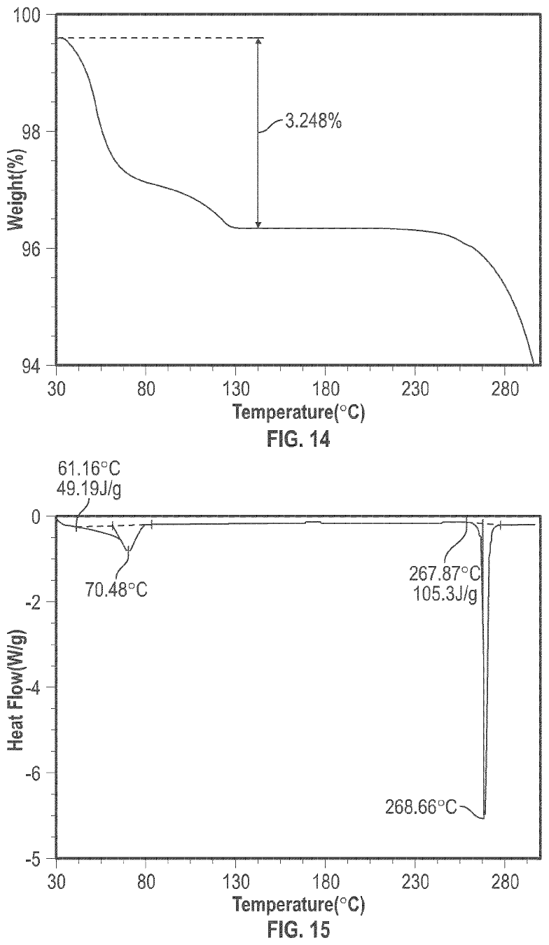

In some embodiments, crystalline Form 5 of plinabulin has substantially the same XRPD pattern as depicted in FIG. 13, with corresponding tabulated peak data shown in Table 5.

TABLE 5
Peak Data of PXRD pattern of crystalline Form 5

| Angle, 2θ | d spacing | Intensity, % |
|---|---|---|
| 8.04 | 10.99 | 100 |
| 8.79 | 10.06 | 2.2 |
| 9.64 | 9.17 | 4.2 |
| 10.94 | 8.08 | 5 |
| 12.15 | 7.28 | 7.2 |
| 13.09 | 6.76 | 10.9 |
| 15.07 | 5.87 | 9.5 |
| 16.04 | 5.52 | 6.9 |
| 16.25 | 5.45 | 6.9 |
| 17.67 | 5.01 | 2.2 |
| 18.76 | 4.73 | 4.4 |
| 19.20 | 4.62 | 4.8 |
| 19.81 | 4.48 | 3.6 |
| 21.84 | 4.07 | 4.1 |
| 23.06 | 3.85 | 6 |
| 23.87 | 3.72 | 11.8 |
| 24.10 | 3.69 | 17.1 |
| 24.49 | 3.63 | 8.8 |
| 25.43 | 3.50 | 3.8 |
| 26.60 | 3.35 | 4.5 |
| 27.91 | 3.19 | 2.6 |
| 28.36 | 3.14 | 4.9 |
| 29.36 | 3.04 | 3.5 |

The crystalline Form 5 may further be characterized by a DSC thermogram substantially the same as that depicted in FIG. 15. As shown in FIG. 15, the crystalline Form 5 has a melting point of about 267° C.; the crystalline Form 5 has a differential scanning calorimetry thermogram with endothermic peaks at about 70° C. and at about 267° C.

### Plinabulin Crystalline Form 6

Some embodiments relate to a crystalline Form 6 of plinabulin and its process of preparation.

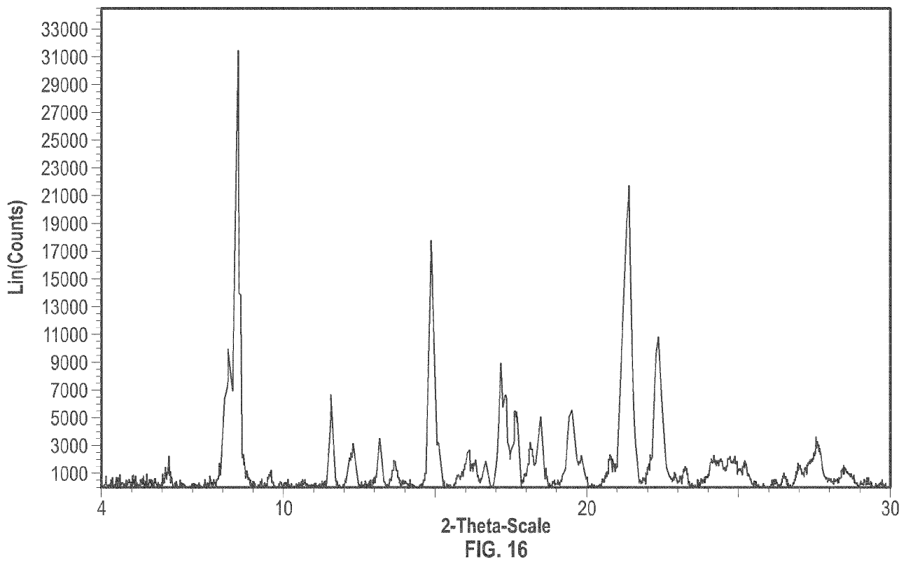

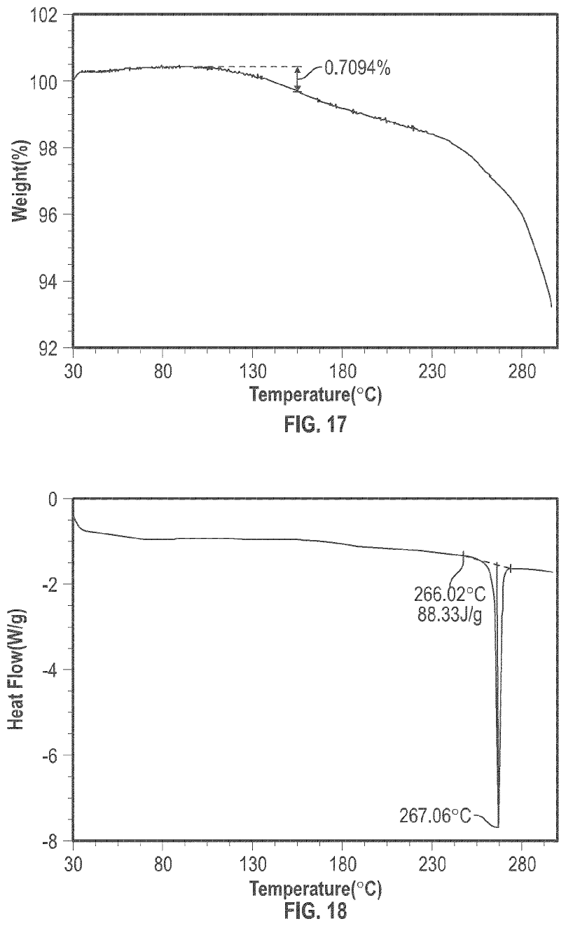

In some embodiments, crystalline Form 6 of plinabulin has substantially the same XRPD pattern as depicted in FIG. 16, with corresponding tabulated peak data shown in Table 6.

TABLE 6
Peak Data of PXRD pattern of crystalline Form 6

| Angle, 2θ | d spacing | Intensity, % |
|---|---|---|
| 6.18 | 14.28 | 8.6 |
| 8.21 | 10.77 | 32.1 |
| 8.49 | 10.41 | 100 |
| 9.56 | 9.25 | 3.8 |
| 11.59 | 7.63 | 21.4 |
| 12.32 | 7.18 | 9.9 |
| 13.20 | 6.70 | 11 |
| 13.69 | 6.46 | 6.6 |
| 14.90 | 5.94 | 57.4 |
| 16.08 | 5.51 | 8.1 |
| 16.68 | 5.31 | 5.7 |
| 17.20 | 5.15 | 27.9 |
| 17.33 | 5.11 | 21 |
| 17.68 | 5.01 | 17.4 |
| 18.19 | 4.87 | 10.5 |
| 18.50 | 4.79 | 16.6 |
| 19.53 | 4.54 | 18.6 |
| 20.84 | 4.26 | 7.1 |
| 21.40 | 4.15 | 69.3 |
| 22.40 | 3.97 | 34.7 |
| 23.30 | 3.81 | 4.9 |
| 24.28 | 3.66 | 8 |
| 24.76 | 3.59 | 6.9 |
| 25.25 | 3.52 | 6.2 |
| 26.57 | 3.35 | 3.2 |
| 27.05 | 3.29 | 5.4 |
| 27.68 | 3.22 | 10.2 |
| 28.55 | 3.12 | 4.9 |

The crystalline Form 6 may further be characterized by a DSC thermogram substantially the same as that depicted in FIG. 18. As shown in FIG. 18, the crystalline Form 6 has a melting point of about 267° C.; the crystalline Form 6 has a differential scanning calorimetry thermogram with endothermic peak at about 267° C.

### Plinabulin Crystalline Form 7

Some embodiments relate to a crystalline Form 7 of plinabulin and its process of preparation.

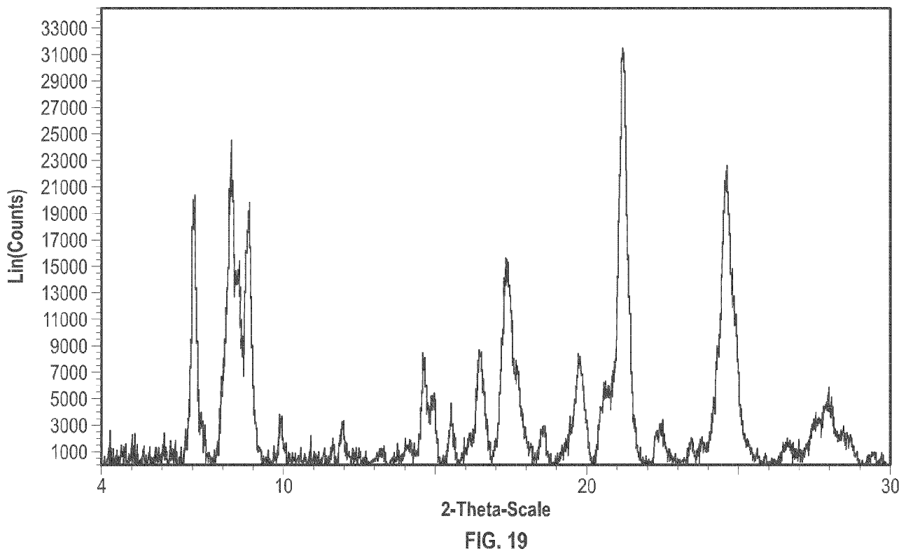

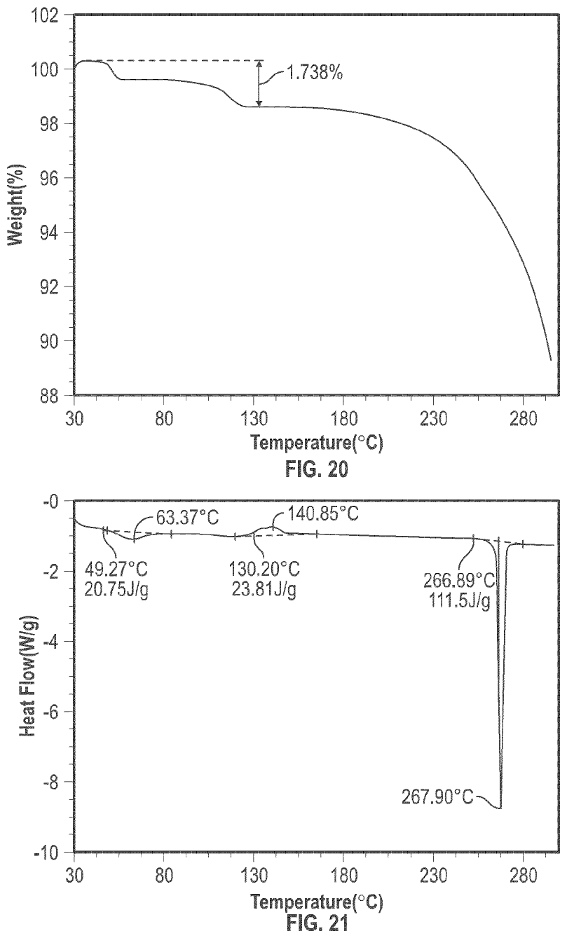

In some embodiments, crystalline Form 7 of plinabulin has substantially the same XRPD pattern as depicted in FIG. 19, with corresponding tabulated peak data shown in Table 7.

TABLE 7
Peak Data of PXRD pattern of crystalline Form 7

| Angle, 2θ | d spacing | Intensity, % |
|---|---|---|
| 6.18 | 14.28 | 8.6 |
| 8.21 | 10.77 | 32.1 |
| 8.49 | 10.41 | 100 |
| 9.56 | 9.25 | 3.8 |
| 11.59 | 7.63 | 21.4 |
| 12.32 | 7.18 | 9.9 |
| 13.20 | 6.70 | 11 |
| 13.69 | 6.46 | 6.6 |
| 14.90 | 5.94 | 57.4 |
| 16.08 | 5.51 | 8.1 |
| 16.68 | 5.31 | 5.7 |
| 17.20 | 5.15 | 27.9 |
| 17.33 | 5.11 | 21 |
| 17.68 | 5.01 | 17.4 |
| 18.19 | 4.87 | 10.5 |
| 18.50 | 4.79 | 16.6 |
| 19.53 | 4.54 | 18.6 |
| 20.84 | 4.26 | 7.1 |
| 21.40 | 4.15 | 69.3 |
| 22.40 | 3.97 | 34.7 |
| 23.30 | 3.81 | 4.9 |
| 24.28 | 3.66 | 8 |
| 24.76 | 3.59 | 6.9 |
| 25.25 | 3.52 | 6.2 |
| 26.57 | 3.35 | 3.2 |
| 27.05 | 3.29 | 5.4 |
| 27.68 | 3.22 | 10.2 |
| 28.55 | 3.12 | 4.9 |

The crystalline Form 7 may further be characterized by a DSC thermogram substantially the same as that depicted in FIG. 21. As shown in FIG. 21, the crystalline Form 7 has a melting point of about 267° C.; the crystalline Form 7 has a differential scanning calorimetry thermogram with endothermic peaks at about 63° C. and at about 267° C.

### Plinabulin Crystalline Form 8

Some embodiments relate to a crystalline Form 8 of plinabulin and its process of preparation.

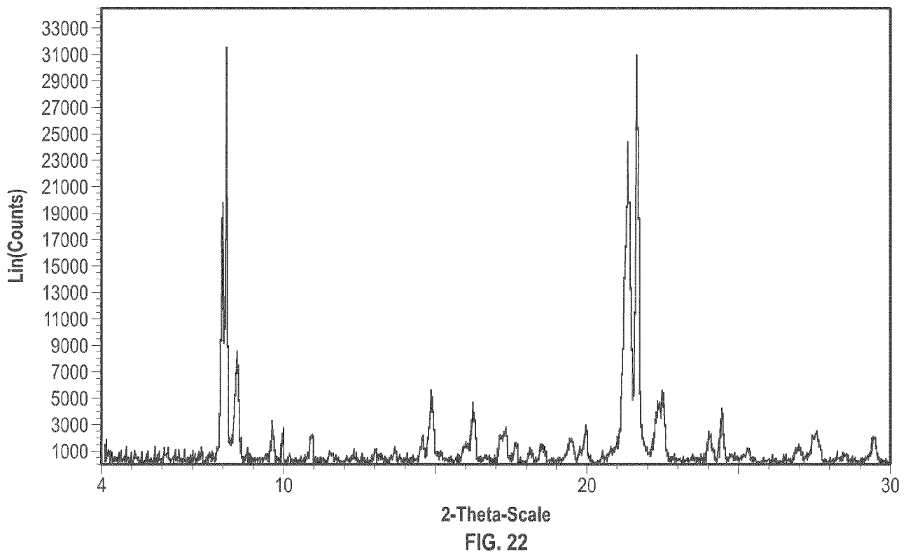

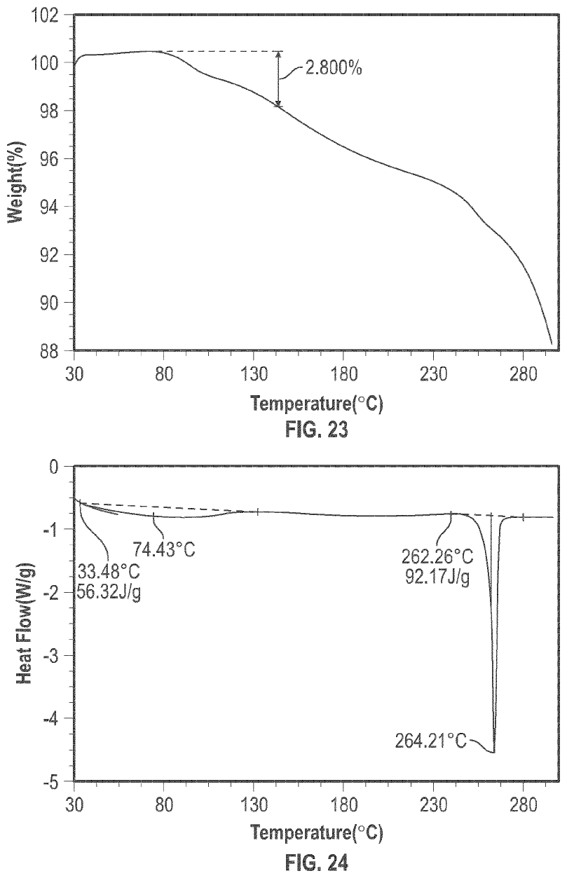

In some embodiments, crystalline Form 8 of plinabulin has substantially the same XRPD pattern as depicted in FIG. 22, with corresponding tabulated peak data shown in Table 8.

TABLE 8
Peak Data of PXRD pattern of crystalline Form 8

| Angle, 2θ | d spacing | Intensity, % |
|---|---|---|
| 6.18 | 14.28 | 8.6 |
| 8.21 | 10.77 | 32.1 |
| 8.49 | 10.41 | 100 |
| 9.56 | 9.25 | 3.8 |
| 11.59 | 7.63 | 21.4 |
| 12.32 | 7.18 | 9.9 |
| 13.20 | 6.70 | 11 |
| 13.69 | 6.46 | 6.6 |
| 14.90 | 5.94 | 57.4 |
| 16.08 | 5.51 | 8.1 |
| 16.68 | 5.31 | 5.7 |
| 17.20 | 5.15 | 27.9 |
| 17.33 | 5.11 | 21 |
| 17.68 | 5.01 | 17.4 |
| 18.19 | 4.87 | 10.5 |
| 18.50 | 4.79 | 16.6 |
| 19.53 | 4.54 | 18.6 |
| 20.84 | 4.26 | 7.1 |
| 21.40 | 4.15 | 69.3 |
| 22.40 | 3.97 | 34.7 |
| 23.30 | 3.81 | 4.9 |
| 24.28 | 3.66 | 8 |
| 24.76 | 3.59 | 6.9 |
| 25.25 | 3.52 | 6.2 |
| 26.57 | 3.35 | 3.2 |
| 27.05 | 3.29 | 5.4 |
| 27.68 | 3.22 | 10.2 |
| 28.55 | 3.12 | 4.9 |

The crystalline Form 8 may further be characterized by a DSC thermogram substantially the same as that depicted in FIG. 24. As shown in FIG. 24, the crystalline Form 8 has a melting point of about 262° C.; the crystalline Form 8 has a differential scanning calorimetry thermogram with endothermic peaks at about 74° C. and at about 264° C.

### Plinabulin Crystalline Form 9

Some embodiments relate to a crystalline Form 9 of plinabulin and its process of preparation.

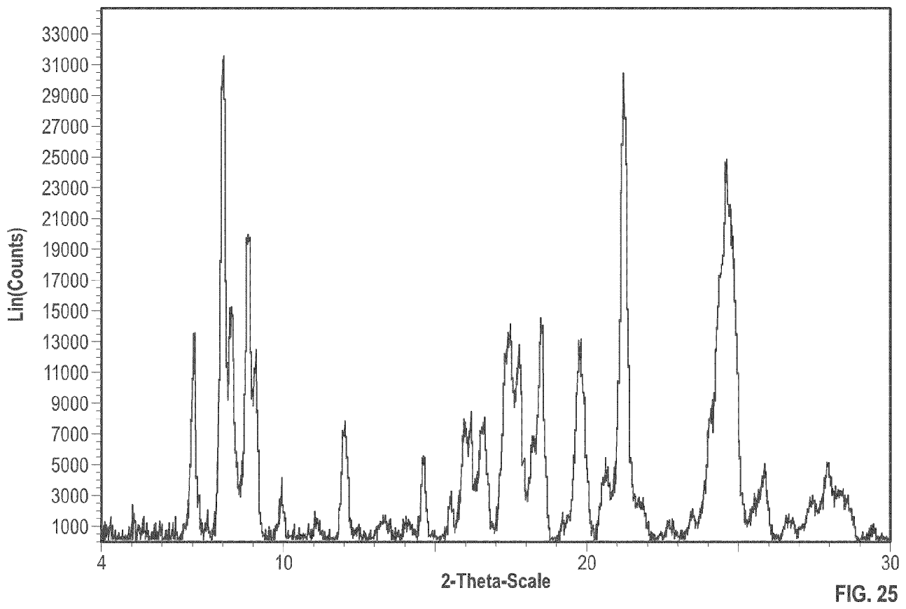

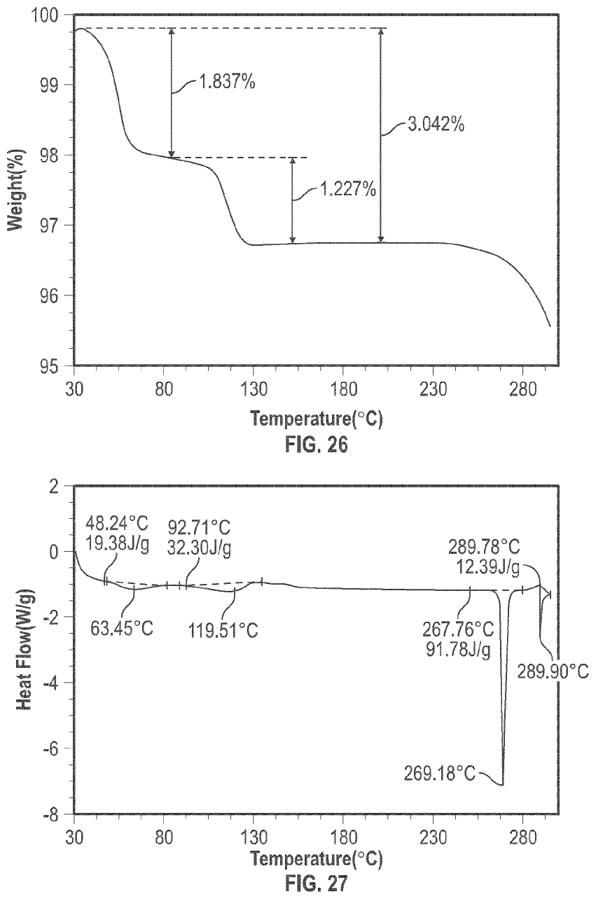

In some embodiments, crystalline Form 9 of plinabulin has substantially the same XRPD pattern as depicted in FIG. 25, with corresponding tabulated peak data shown in Table 9.

TABLE 9
Peak Data of PXRD pattern of crystalline Form 9

| Angle, 2θ | d spacing | Intensity, % |
|---|---|---|
| 7.03 | 12.57 | 43 |
| 8.00 | 11.04 | 100 |
| 8.27 | 10.68 | 48.3 |
| 8.84 | 9.99 | 63.1 |
| 9.08 | 9.74 | 38.4 |
| 9.94 | 8.89 | 12.8 |
| 11.11 | 7.96 | 4.4 |
| 12.03 | 7.35 | 24.6 |
| 13.32 | 6.64 | 5 |
| 14.07 | 6.29 | 5.1 |
| 14.64 | 6.05 | 17.4 |
| 15.56 | 5.69 | 10 |
| 16.02 | 5.53 | 24.2 |
| 16.21 | 5.46 | 26.6 |
| 16.57 | 5.35 | 24.4 |
| 17.45 | 5.08 | 43.7 |
| 17.74 | 4.99 | 39.4 |
| 18.24 | 4.86 | 21.5 |
| 18.50 | 4.79 | 46 |
| 19.78 | 4.49 | 41.2 |
| 20.63 | 4.30 | 17 |
| 21.23 | 4.18 | 96.5 |
| 21.80 | 4.07 | 8 |
| 22.72 | 3.91 | 4.1 |
| 23.50 | 3.78 | 6.5 |
| 24.64 | 3.61 | 78.6 |
| 25.88 | 3.44 | 14.6 |
| 26.68 | 3.34 | 4.8 |
| 27.46 | 3.25 | 9.3 |
| 27.99 | 3.19 | 16 |
| 29.45 | 3.03 | 3.2 |

The crystalline Form 9 may further be characterized by a DSC thermogram substantially the same as that depicted in FIG. 27. As shown in FIG. 27, the crystalline Form 9 has a melting point of about 267° C.; the crystalline Form 9 has a differential scanning calorimetry thermogram with endothermic peaks at about 63° C., about 119° C., about 267° C., and about 289° C.

### Conversion of Plinabulin Polymorph Forms

The plinabulin monohydrate (Form 1) is the most stable polymorph among the nine polymorph forms identified. The plinabulin monohydrate (Form 1) remains stable during the drying process and under humidity-based stability studies (stable to drying at 50° C. under vacuum over the weekend, no change in solid form on exposure to humidity higher than 95% RH for 13 days).

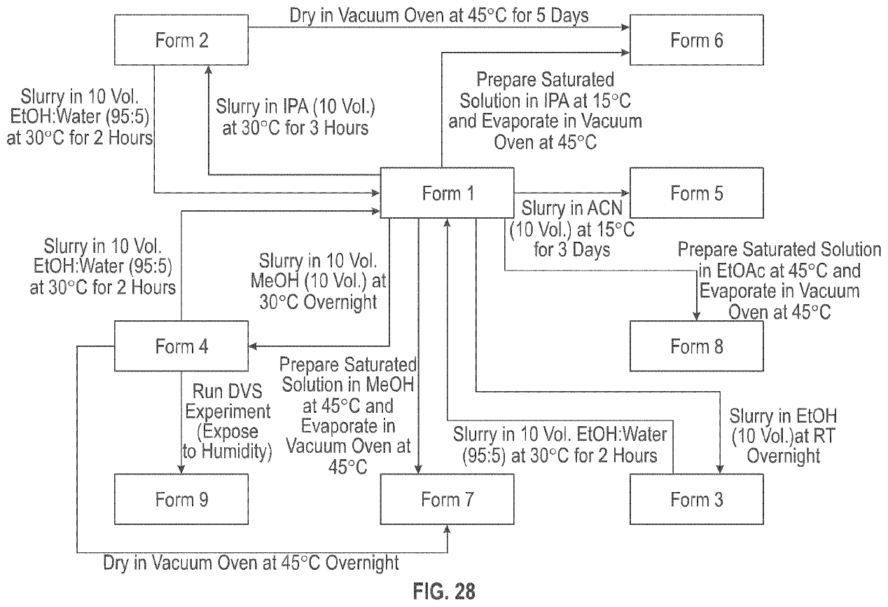

FIG. 28 shows how Form 1 can be converted to the other eight forms of plinabulin polymorph forms. For example, the plinabulin monohydrate (Form 1) can be converted to Form 2 by slurring Form 1 in isopropyl alcohol (about 10 times the volume of Form 1) at 30° C. for 3 hours; Form 1 can be converted to Form 3 by slurring Form 1 in ethanol (about 10 times the volume of Form 1) at room temperature for overnight; Form 1 can be converted to Form 4 by slurring Form 1 in methanol (about 10 times the volume of Form 1) at 30° C. for overnight; Form 1 can be converted to Form 5 by slurring Form 1 in in acetonitrile (ACN) at 30° C. and stirring for 3 days; Form 1 can be converted to Form 6 by preparing a Form 1 saturated isopropyl alcohol solution at 15° C. and then evaporate in vacuum over at 45° C.; Form 1 can be converted to Form 7 by preparing a Form 1 saturated methanol solution at 45° C. and then evaporate in vacuum over at 45° C.; Form 1 can be converted to Form 8 by preparing a Form 1 saturated ethyl acetate (EtOAc) solution at 45° C. and then evaporate in vacuum over at 45° C.; and Form I can be converted to Form 9 by first converting Form 1 to Form 4 and then exposing Form 4 to moisture.

FIG. 28 also shows how other forms can be converted to Form 1. For example, Forms 2, 3, and 4 can be converted to Form 1 by slurrying these forms in a mixture of ethanol and water (95:5 by volume) (the volume of ethanol and water mixture is about 10times of the staring polymorph forms) at 30° C. for 2 hours; Form 2 can be converted to Form 6 by drying it in vacuum oven at 45° C. for 5 days; Form 4 can be converted to Form 7 by drying it in vacuum oven at 45° C. overnight; and Form 4 can be converted to Form 9 by exposing it to high humidity.

### Administration and Pharmaceutical Compositions

Some embodiments include pharmaceutical compositions comprising the plinabulin polymorph described herein and a pharmaceutically acceptable carrier. Such a composition can be administered to a subject as part of a therapeutic treatment.

In some embodiments, the composition can further include one or more pharmaceutically acceptable diluents. In some embodiments, the pharmaceutically acceptable diluent can include Kolliphor® (Polyoxyl 15 hydroxystearate). In some embodiments, the pharmaceutically acceptable diluent can include propylene glycol. In some embodiments, the pharmaceutically acceptable diluents can include kolliphor and propylene glycol. In some embodiments, the pharmaceutically acceptable diluents can include kolliphor and propylene glycol, wherein the kolliphor is about 40% by weight and propylene glycol is about 60% by weight based on the total weight of the diluents. In some embodiments, the composition can further include one or more other pharmaceutically acceptable excipients.

Standard pharmaceutical formulation techniques can be used to make the pharmaceutical compositions described herein, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of Plinabulin polymorph or pharmaceutically acceptable salts thereof; and (b) a pharmaceutically acceptable carrier, diluent, excipient or combination thereof.

Other embodiments include co-administering plinabulin polymorph and an additional therapeutic agent in separate compositions or the same composition. Thus, some embodiments include a first pharmaceutical compositions comprising: (a) a safe and therapeutically effective amount of plinabulin polymorph or pharmaceutically acceptable salts thereof and (b) a pharmaceutically acceptable carrier, diluent, excipient or combination thereof; and a second pharmaceutical composition comprising: (a) a safe and therapeutically effective amount of an additional therapeutic agent and (b) a pharmaceutically acceptable carrier, diluent, excipient or combination thereof. Some embodiments include a pharmaceutical composition comprising: (a) a safe and therapeutically effective amount of plinabulin polymorph or pharmaceutically acceptable salts thereof; (b) a safe and therapeutically effective amount of an additional therapeutic agent; and (c) a pharmaceutically acceptable carrier, diluent, excipient or combination thereof.

Administration of the pharmaceutical compositions described herein can be via any of the accepted modes of administration for agents that serve similar utilities including, but not limited to, orally, sublingually, buccally, subcutaneously, intravenously, intranasally, topically, transdermally, intradermally, intraperitoneally, intramuscularly, intrapulmonarilly, vaginally, rectally, or intraocularly. Oral and parenteral administrations are customary in treating the indications that are the subject of the preferred embodiments.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The compositions described herein are preferably provided in unit dosage form. As used herein, a “unit dosage form” is a composition containing an amount of a compound or composition that is suitable for administration to an animal, preferably a mammalian subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, although a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, sublingual, buccal, nasal, rectal, topical (including transdermal and intradermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropics, surface-active agents, and encapsulating substances. Optional pharmaceutically-active materials may be included, which do not substantially interfere with the activity of the compound or composition. The amount of carrier employed in conjunction with the compound or composition is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules (e.g., liquid gel capsule and solid gel capsule), granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, sucrose, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject composition is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium (EDTA), although other chelating agents may also be used in place or in conjunction with it.

For topical use, creams, ointments, gels, solutions or suspensions, etc., containing the composition disclosed herein are employed. Topical formulations may generally be comprised of a pharmaceutical carrier, co-solvent, emulsifier, penetration enhancer, preservative system, and emollient.

For intravenous administration, the compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

In some embodiments, a single dose of plinabulin polymorph or other therapeutic agent may be from about 5 mg/m² to about 150 mg/m² of body surface area, from about 5 mg/m² to about 100 mg/m² of body surface area, from about 10 mg/m² to about 100 mg/m² of body surface area, from about 10 mg/m² to about 80 mg/m² of body surface area, from about 10 mg/m² to about 50 mg/m² of body surface area, from about 10 mg/m² to about 40 mg/m² of body surface area, from about 10 mg/m² to about 30 mg/m² of body surface area, from about 13.5 mg/m² to about 100 mg/m² of body surface area, from about 13.5 mg/m² to about 80 mg/m² of body surface area, from about 13.5 mg/m² to about 50 mg/m² of body surface area, from about 13.5 mg/m² to about 40 mg/m² of body surface area, from about 13.5 mg/m² to about 30 mg/m² of body surface area, from about 15 mg/m² to about 80 mg/m² of body surface area, from about 15 mg/m² to about 50 mg/m² of body surface area, or from about 15 mg/m² to about 30 mg/m² of body surface area. In some embodiments, a single dose of plinabulin polymorph or other therapeutic agent may be from about 13.5 mg/m² to about 30 mg/m² of body surface area. In some embodiments, a single dose of plinabulin polymorph or other therapeutic agent may be about 5 mg/m², about 10 mg/m², about 12.5 mg/m², about 13.5 mg/m², about 15 mg/m², about 17.5 mg/m², about 20 mg/m², about 22.5 mg/m², about 25 mg/m², about 27.5 mg/m², about 30 mg/m², about 40 mg/m², about 50 mg/m², about 60 mg/m², about 70 mg/m², about 80 mg/m², about 90 mg/m², or about 100 mg/m², of body surface area.

In some embodiments, a single dose of plinabulin polymorph or other therapeutic agent may be from about 5 mg to about 300 mg, from about 5 mg to about 200 mg, from about 7.5 mg to about 200 mg, from about 10 mg to about 100 mg, from about 15 mg to about 100 mg, from about 20 mg to about 100 mg, from about 30 mg to about 100 mg, from about 40 mg to about 100 mg, from about 10 mg to about 80 mg, from about 15 mg to about 80 mg, from about 20 mg to about 80 mg, from about 30 mg to about 80 mg, from about 40mg to about 80 mg, from about 10 mg to about 60 mg, from about 15 mg to about 60 mg, from about 20 mg to about 60 mg, from about 30 mg to about 60 mg, or from about 40 mg to about 60 mg, In some embodiments, a single dose of plinabulin polymorph or other therapeutic agent may be from about 20 mg to about 60 mg, from about 27 mg to about 60 mg, from about 20mg to about 45 mg, or from about 27 mg to about 45 mg. In some embodiments, a single dose of plinabulin polymorph or other therapeutic agent may be about 5 mg, about 10 mg, about 12.5 mg, about 13.5 mg, about 15 mg, about 17.5 mg, about 20 mg, about 22.5 mg, about 25mg, about 27 mg, about 30 mg, about 40 mg, about 50 mg, about 60 mg, about 70 mg, about 80 mg, about 90 mg, about 100 mg, about 125 mg, about 150 mg, or about 200 mg.

The administration period can be a multi-week treatment cycle as long as the tumor remains under control and the regimen is clinically tolerated. In some embodiments, a single dosage of plinabulin polymorph or other therapeutic agent can be administered once a week, and preferably once on each of day I and day 8 of a three-week (21 day) treatment cycle. In some embodiments, a single dosage of plinabulin polymorph or other therapeutic agent can be administered once a week, twice a week, three times per week, four times per week, five times per week, six times per week, or daily during a one-week, two-week, three-week, four-week, or five-week treatment cycle. The administration can be on the same or different day of each week in the treatment cycle.

The treatment cycle can be repeated as long as the regimen is clinically tolerated. In some embodiments, the treatment cycle is repeated for n times, wherein n is an integer in the range of 2 to 30. In some embodiments, n is 2, 3, 4, 5, 6, 7, 8, 9, or 10. In some embodiments, a new treatment cycle can occur immediately after the completion of the previous treatment cycle. In some embodiments, a new treatment cycle can occur a period of time after the completion of the previous treatment cycle.

In some embodiments, the compositions described herein can be used in combination with other therapeutic agents. In some embodiments, the compositions described herein can be administered or used in combination with treatments such as chemotherapy, radiation, and biologic therapies.

### EXAMPLES

### Example 1

A sample of plinabulin compound was stirred in ethanol and heated to reflux. Ethanol was added in portions to maintain reflux until the entire sampled dissolved to give a clear yellow solution. A total of 124.7 g of ethanol were required to completely dissolve the sample at reflux. The solution was then allowed to cool and monitored for precipitation. A precipitate was observed when the solution was at 49° C. The mixture was reheated to reflux providing a clear yellow solution. The hot solution was transferred to a larger Erlenmeyer flask with a 9.45 g ethanol rinse (to mimic a hot filtration). To this refluxing solution was added 6.6 g of water (approximately 5% water in ethanol). This solution was allowed to cool slowly with stirring. A precipitate was observed when the solution cooled to 70° C. At this point additional water (128.6 g) was added slowly causing a large amount of solid to precipitate. The solution was allowed to cool to room temperature with stirring. The solids were filtered at 17° C. and washed three times with 20 g of water. A total of 134 g of water was added to the filtrate causing a hazy solution, an insufficient amount of solid to filter. An additional 137 g of water was added in an attempt to precipitate additional product but no additional solid was recoverable. The solid was dried at 40-45° C. for 3 days to give 4.73 g, 97.5% recovery. Analysis by XRPD showed the product to be plinabulin monohydrate (Form 1). Analysis by Karl Fischer showed the moisture level was 4.9%.

Analysis by KF after drying at 45° C. under vacuum for an additional 70 hours indicated the moisture level had decreased to 4.1%. The XRPD of this sample indicated that it was plinabulin monohydrate (Form 1). This sample was placed in a glove bag in an open container with an open container of water and the moisture level monitored. After 4 hours the moisture level was measured as 5.0%. After 18.5 hours the moisture level was measured as 4.8% and after 51 hours the KF result was 4.9%.

### Example 2

A sample of plinabulin compound (4.92 g) was stirred in ethanol (147.6 g) and heated to reflux (fully soluble at 75° C.). The solution was then allowed to cool and monitored for precipitation. A precipitate was observed when the solution was at 48° C. The mixture was re-heated to reflux providing a clear yellow solution. To the hot solution was added 295 g of water (approximately twice the mass of ethanol) allowing the mixture to cool during the addition. A precipitate was observed after charging approximately 150 ml of water with the temperature at 48° C. This solution was allowed to cool to room temperature. The solids were filtered and washed three times with 20 g of water. The solid was dried at 40-45° C. for 2.5 days to give 4.82 g, 98.0% recovery. Analysis by XRPD showed the product to be a mix of plinabulin monohydrate (Forms 1) (major) and anhydrous plinabulin (Form 3) (minor). Analysis by Karl Fischer showed the moisture level was 5.0%. Analysis by KF after drying at 45° C. under vacuum for an additional 70 hours indicated the moisture level had decreased to 4.4%. The XRPD of this sample indicates it was essentially unchanged, a mixture of Forms 1 and 3 with additional peaks (at around 12.26°, 15.19° and 28.79° 2θ degrees). This sample was placed in a glove bag in an open container with an open container of water and the moisture level monitored. After 4 hours the moisture level was measured as 5.0%. After 18.5 hours the moisture level was measured as 4.9% and after 51 hours the KF result was 5.1%.

### Example 3

A sample of plinabulin compound was dissolved in 1,2-propanediol with vigorous stirring or mild heating (50° C.). After 4 hours, the sample showed peaks associated with Form 3. After stirring over the weekend, the sample was slurried in water and converted entirely to plinabulin monohydrate (Form 1) with no peaks associated with crystalline Form 3 present in the XRPD scans. A similar result was seen for the experiment in ethanol/water, with very small peaks seen for Form 3 at 1 hour and 4 hours and only Form 1 observed after 66 hours.

### Example 4

In a reprocessing procedure, a plinabulin compound (Form 3) was dissolved in ethanol at a ratio of 1:25 (by weight) at reflux. This solution was filtered at a temperature that was higher 50° C. and the filtrate was combined with an equal mass of water to afford the product. It may be desirable to reheat the polish-filtered ethanol solution prior to addition of the entire amount of water and add approximately 5% water (relative to ethanol) and stir this solution at approximately 70° C. to ensure conversion to Form 1. Additional water can then be added and the mixture cooled to isolate the product by filtration. The sample can be dried for an extended period to lower the moisture content as measured by Karl Fisher analysis. One sample was dried for three days resulting in a KF analysis of 4.9% moisture. Drying for an additional three days lowered the KF result to 4.1%.

Exposing the dry product to a humid environment raised the moisture level to approximately 5% where it appeared to remain stable. One sample with 4.1% moisture was exposed to an open container of water in a glove bag for 4 hours, raising the moisture level to 5.0%. After an additional 14.5 hours in this environment the moisture level in the sample was measured as 4.8%. After a total of 51 hours the KF result was 4.9%.

### Example 5

A batch of plinabulin having a KF analysis of about 3.1% for water content was added to a mixture of kolliphor (40% wt) and propylene glycol (60% wt). Insoluble particles were formed in the solution, and it was determined that the insoluble particles were anhydrous plinabulin (Form 3). The batch was reprocessed according to the steps described in FIG. 29 to form the plinabulin monohydrate (Form 1). Analysis by KF showed that the water content of the reprocessed plinabulin was about 5.1%, which is consistent with the theoretical water content for the monohydrate. The plinabulin monohydrate (Form 1) dissolved completely in a mixture of kolliphor (40% wt) and propylene glycol (60% wt) and no insoluble particles were formed in the solution. Therefore, the plinabulin monohydrate (Form 1) showed better solubility than plinabulin compositions that contain anhydrous plinabulin (Form 3).

### Example 6

The plinabulin monohydrate (Form 1 crystalline) was characterized by XRPD (crystalline, FIG. 1), optical microscopy (FIG. 2), DSC (FIG. 3A), TGA (FIG. 3B), and KF.

The DSC data were collected using a TA Instruments Q10 DSC. Typically, samples (˜2 mg) were placed in hermetic alodined aluminum sample pans and scanned from 30 to 300° C. at a rate of 10° C./min under a nitrogen purge of 50 mL/min. The TGA data were collected using a TA Instruments TGA Q500. Typically, samples (˜10 mg) were placed in an open, pre-tared aluminum sample pan and scanned from 30 to 300° C. at a rate of 10° C./min using a nitrogen purge at 60 mL/min. The X-ray powder diffraction patterns were obtained using a Bruker D8 Advance equipped with a Cu Kα radiation source (1.54° A), a 9-position sample holder and a LYNXEYE Super Speed Detector. Typically, the duration of each scan was 180 seconds and the 2θ range was 4 to 40°. Samples were placed on zero-background, silicon plate holders. Samples were analyzed using an Aquadyne DVS-2 gravimetric water sorption analyzer. The relative humidity was adjusted between 2-95% and the weight of sample was continuously monitored and recorded.

The XRPD showed that the material is crystalline. The DSC data showed a broad endotherm (peak max at 141° C., likely water loss), a small exothermic event (peak max 164° C.) and a sharp endothermic event (peak max 268° C.). TGA indicated loss of 5.26% of mass at about 130° C. (likely water loss). KF analysis also showed that the material contains water at 5.25 weight %.

A sample of plinabulin monohydrate (Form 1) was placed in a vacuum oven at 50° C. overnight and over the weekend. The sample remained stable during the drying studies and no change in weight occurred during the drying process.

A DVS experiment was run on the plinabulin monohydrate (Form 1). The sample gained ˜0.1% mass at 95% RH which was lost on drying to 0% RH without hysteresis. The post-DVS sample was analyzed by XRPD which confirmed that no transformation had taken place. FIG. 3C shows the DVS isotherm plot. The XRPD pattern of the pre-DVS sample (Form 1 plinabulin monohydrate) overlays with the XRPD patterns of the post-DVS sample (Form 1 plinabulin monohydrate).

The plinabulin monohydrate (Form 1) sample was placed in a high humidity environment for 13 days and remained stable with no change in mass. The pre-DVS and post-DVS samples showed no change in the XRPD pattern.

The DVS test of Form 2 solids showed that the Form 2 sample lost about 7% mass at about 80% RH as shown in FIG. 6C, and the post-DVS sample differed from the pre-DVS sample based on the XRPD pattern analysis.

The DVS test of Form 3 solids showed that the Form 3 sample lost about 0.2% mass at 90% RH as shown in FIG. 9C, and the post-DVS sample differed from the pre-DVS sample based on the XRPD pattern analysis.

The DVS test of Form 4 solids showed that the Form 3 sample lost mass in the test, and the DVS isotherm plot of Form 4 is shown in FIG. 12C. The post-DVS sample differed from the pre-DVS sample based on the XRPD pattern analysis.

The plinabulin monohydrate (Form 1) remained stable during the DVS and drying tests. In comparison, other polymorph forms were unstable and showed weight change during the DVS tests. The test results have shown that Form 1 is more stable than the other polymorph forms.

### Example 7

A plinabulin compound was slurried in 15 different solvents/solvent mixtures as shown in Table 9 at 15° C. and 45° C. for 3 days for gravimetric solubility measurement. About 70 mg of solid was added to a vial for each experiment followed by addition of 0.7 mL of the respective solvents. Next, the slurry was centrifuged and the supernatant was added to pre-weighed vials and evaporated to dryness under vacuum. The vials with remaining solids were weighed again to calculate the solubility. The compound is highly soluble in THF and had moderate to low solubility in the other solvents tested. The solubility data is shown in Table 10.

TABLE 10
Gravimetric solubility data of plinabulin monohydrate in various
solvents.

| Sample | | Solubility at | Solubility at |
|---|---|---|---|
| No. | Solvent | 15° C. in mg/mL | 45° C. in mg/mL |
| 1 | Heptane | <1 | 3 |
| 2 | Toluene | 1 | 7 |
| 3 | MTBE | <1 | 5 |
| 4 | EtOAc | 3 | 8 |
| 5 | THF | 40 | 96 |
| 6 | IPA | 1 | 7 |
| 7 | Acetone | 11 | 20 |
| 8 | EtOH | 5 | 13 |
| 9 | MeOH | 5 | 13 |
| 10 | ACN | 3 | 7 |
| 11 | Water | <1 | <1 |
| 12 | MEK | 11 | 23 |
| 13 | DCM | 2 | N/A |
| 14 | Acetone:water (95:5) | 6 | 16 |
| 15 | EtOH:water (95:5) | 4 | 13 |

During gravimetric solubility analysis, the plinabulin monohydrate (Form 1) was slurried in various solvents at 15° C. and at 45° C. After centrifugation, the solids obtained were analyzed as wet cake by XRPD. The samples which were not Form 1 were dried in a vacuum oven and reanalyzed by XRPD. Forms 2, 3, 4, 5, 6, and 7 were observed. These results are shown in Table 11.

TABLE 11
XRPD results of slurry experiments in various solvents before
and after drying

| | | 15° C. | 45° C. | 15° C. | 45° C. |
|---|---|---|---|---|---|
| | | slurry | slurry | slurry | slurry |
| | | wet | wet | dry | dry |
| No. | Solvent | XRPD | XRPD | XRPD | XRPD |
| 1 | Heptane | Form 1 | Form 1 | N/A | N/A |
| 2 | Toluene | Form 1 | Form 1 | N/A | N/A |
| 3 | MTBE | Form 1 | Form 1 | N/A | N/A |
| 4 | EtOAc | Form 1 | Form | N/A | Still Form |
| | | | 1 + 3 | | 1 + 3 |
| 5 | THF | Form 1 | Form | N/A | Still Form |
| | | | 1 + 3 | | 1 + 3 |
| 6 | IPA | Form 2 | Form | Form 6 | Form 6 + 2 |
| | | | 1 + 2 | | |
| 7 | Acetone | Form | Form 3 | Still Form | Still Form 3 |
| | | 1 + 3 | | 1 + 3 | |
| 8 | EtOH | Form 3 | Form 3 | Still Form 3 | Still Form 3 |
| 9 | MeOH | Form | Form 4 | Still Form | Form 7 |
| | | 1 + 4 | | 1 + 4 | |
| 10 | ACN | Form 5 | Form 3 | Still Form 5 | N/A |
| 11 | Water | Form 1 | Form 1 | N/A | N/A |
| 12 | MEK | Form 1 | Form 3 | N/A | N/A |
| 13 | DCM | Form 1 | N/A | N/A | N/A |
| 14 | Acetone:water | Form 1 | Form 1 | N/A | N/A |
| | (95:5) | | | | |
| 15 | EtOH:water | Form 1 | Form 1 | N/A | N/A |
| | (95:5) | | | | |

Evaporation crystallization experiments were setup by evaporating (at room temperature) solutions of the plinabulin monohydrate in various solvents. These solutions were obtained during the gravimetric solubility analysis at 15° C. and at 45° C. and were of different concentrations. The solids obtained were analyzed by XRPD. Form 8 was observed. These results are shown in Table 12.

TABLE 12
XRPD results of evaporation crystallization experiments

| | | Evaporation of | Evaporation of |
|---|---|---|---|
| | | 15° C. solubility | 45° C. solubility |
| No. | Solvent | samples | samples |
| 1 | Heptane | Not enough solids | Not enough solids |
| 2 | Toluene | Not enough solids | Not enough solids |
| 3 | MTBE | Not enough solids | Not enough solids |
| 4 | EtOAc | Not enough solids | Form 8 |
| 5 | THF | Form 6 | Form 8 |
| 6 | IPA | Not enough solids | Form 6 |
| 7 | Acetone | Form 3 + 6 | Form 8 |
| 8 | EtOH | Not enough solids | Form 3 |
| 9 | MeOH | Mostly amorphous | Form 7 |
| | | w/Form 8 peak | |
| 10 | ACN | Not enough solids | Form 3 |
| 11 | Water | Not enough solids | Not enough solids |
| 12 | MEK | Form 3 + 6 | Form 3 |
| 13 | DCM | Not enough solids | N/A |
| 14 | Acetone:water | Form 8 | Form 8 |
| | (95:5) | | |
| 15 | EtOH:water | Low Crystallinity | Form 1 |
| | (95:5) | Form 6 | |

Six cooling crystallization experiments and one slurry experiment were carried out. The results of these experiments are shown in Table 13.

TABLE 13
XRPD results of cooling crystallization experiments

| | Solvent | Procedure | XRPD |
|---|---|---|---|
| 1 | THF | 100 mg solids dissolved in 10 vol. | Form |
| | | solvent at 60° C. Cooled naturally to RT | 1 + 5 |
| | | and stirred for 2 hours. Cooled to 5° C. | |
| | | using a chiller and stirred for 3 hours. | |
| | | Crystallized. | |
| 2 | THF:acetone | 100 mg solids dissolved in 16 vol. | Form 1 |
| | 1:1 | solvent at 60° C. Cooled naturally to RT | |
| | | and stirred for 2 hours. Cooled to 5° C. | |
| | | using a chiller and stirred overnight. | |
| | | Crystallized. | |
| 3 | THF:EtOH | 100 mg solids dissolved in 11 vol. | Form 3 |
| | 1:1 | solvent at 60° C. Cooled naturally to RT | |
| | | and stirred for 2 hours. Cooled to 5° C. | |
| | | using a chiller and stirred for 2 hours. | |
| | | Crystallized. | |
| 4 | THF:MeOH | 100 mg solids dissolved in 8 vol. | Form 4 |
| | 1:1 | solvent at 60° C. Cooled naturally to RT | |
| | | and stirred for 2 hours. Cooled to 5° C. | |
| | | using a chiller. Crystallized at 12° C. | |
| 5 | THF:water | 100 mg solids did not dissolve in 20 | Form 1 |
| | 1:1 | vol. solvent at 60° C. Cooled naturally | |
| | | to RT and stirred overnight. | |
| 6 | THF:MEK | 100 mg solids dissolved in 20 vol. | Form 1 |
| | (methyl | solvent at 60° C. Cooled naturally to RT | |
| | ethyl | and stirred for 2 hours. Cooled to 5° C. | |
| | ketone) | using a chiller and stirred for 2 hours. | |
| | 1:1 | Crystallized. | |

### Example 8

The various crystalline forms were tested for conversion to other forms at a 200 mg scale.

A Form 3 scale up experiment was carried out at the 200 mg scale by slurrying Form 1 solids in 2 mL ethanol at room temperature overnight to produce Form 3.

A Form 2 scale up experiment was carried out at the 200 mg scale by slurrying Form 1 solids in 2 mL isopropyl alcohol at 30° C. overnight.

A Form 4 scale up experiment was carried out at the 200 mg scale by slurrying Form 1 solids in 2 mL MeOH at 30° C. overnight.

Form 3 solids were slurried in a 95:5 EtOH: water mixture at 30° C. for 2 hours and analyzed by XRPD to show conversion to Form 1.

Form 2 solids were slurried (2071-16-1) in a 95:5 EtOH: water mixture at 30° C. for 2 hours and analyzed by XRPD to show conversion to Form 1.

Form 4 solids were slurried in a 95:5 EtOH: water mixture at 30° C. for 2 hours and analyzed by XRPD to show conversion to Form 1.

The plinabulin monohydrate (Form 1) remained stable when dried at 50° C. under vacuum over the weekend. There was no change in solid form when form 1 was exposed to high humidity (>95% RH) for 13 days. Form 1 was shown to be stable during the manufacturing process including the drying process. Form 1 was also stable under various humidity conditions. On the other hand, the crystalline forms converted to Form I monohydrate when exposed to moisture. Therefore, Form 1 is the most stable crystalline form and the most viable form for manufacturing process.

1-53. (canceled)

54. A composition, comprising:
plinabulin and water in a ratio of about 1:1; and
one or more non-aqueous pharmaceutically acceptable diluents.

55. The composition of claim 54, wherein the pharmaceutically acceptable diluents comprise polyoxyl 15 hydroxystearate and propylene glycol.

56. The composition of claim 54, wherein the pharmaceutically acceptable diluents comprise about 40% polyoxyl 15 hydroxystearate and about 60% propylene glycol.

57. The composition of claim 54, comprising no more than about 1% by weight of impurities based on the total weight of non-solvent molecules in the composition, wherein the composition comprises less than 1% methanol.

58. A composition, comprising:
about 38 mg plinabulin;
about 2 mg water; and
one or more non-aqueous pharmaceutically acceptable diluents.

59. The composition of claim 58, wherein the pharmaceutically acceptable diluents comprise polyoxyl 15 hydroxystearate and propylene glycol.

60. The composition of claim 58, wherein the pharmaceutically acceptable diluents comprise about 40% polyoxyl 15 hydroxystearate and about 60% propylene glycol.

61. The composition of claim 58, comprising no more than about 1% by weight of impurities based on the total weight of non-solvent molecules in the composition, wherein the composition comprises less than 1% methanol.

62. The composition of claim 58, consisting of:
about 38 mg plinabulin;
about 2 mg water; and
pharmaceutically acceptable diluents consisting of about 40% polyoxyl 15 hydroxystearate and about 60% propylene glycol.

63. A method of treatment, comprising:
diluting the composition of claim 54 in a second pharmaceutically acceptable diluent; and
intravenously administering the diluted composition to a subject.

64. The method of claim 63, wherein the second pharmaceutically acceptable diluent comprises a dextrose solution.

65. A method of treatment, comprising:
diluting the composition of claim 56 in a second pharmaceutically acceptable diluent; and
intravenously administering the diluted composition to a subject.

66. The method of claim 65, wherein the second pharmaceutically acceptable diluent comprises a dextrose solution.

67. A method of treatment, comprising:
diluting the composition of claim 57 in a second pharmaceutically acceptable diluent; and
intravenously administering the diluted composition to a subject.

68. A method of treatment, comprising:
diluting the composition of claim 58 in a second pharmaceutically acceptable diluent; and
intravenously administering the diluted composition to a subject.

69. The method of claim 68, wherein the second pharmaceutically acceptable diluent comprises a dextrose solution.

70. A method of treatment, comprising:
diluting the composition of claim 61 in a second pharmaceutically acceptable diluent; and
intravenously administering the diluted composition to a subject.

71. A method of treatment, comprising:
diluting the composition of claim 62 in a second pharmaceutically acceptable diluent; and
intravenously administering the diluted composition to a subject.

72. The method of claim 71, wherein the second pharmaceutically acceptable diluent comprises a dextrose solution.
